(19) United States
(12) Patent Application Publication
(10) Pub. No.: US 2026/0363627 A1
(43) Pub. Date: Nov. 19, 2026

(54) PROGNOSIS PREDICTION

(71) Applicant: GrayMatters Health Ltd., Haifa (IL)

(72) Inventors: Dmitri LAVRO, Kfar Vradim (IL); Yaakov STERN, Sde Yaakov (IL); Shai ATTIA, Shimshit (IL); Oded Shlomo KRAFT-OZ, Haifa (IL); Ran COHEN, Tel-Aviv (IL)

(73) Assignee: GrayMatters Health Ltd., Haifa (IL)

(21) Appl. No.: 19/139,413

(22) PCT Filed: Dec. 14, 2023
(86) PCT No.: PCT/IL2023/051275

§ 371 Date: Jun. 16, 2025

Related U.S. Application Data
(60) Provisional application No. 63/433,025, filed on Dec. 16, 2022.

(51) Int. Cl. A61B 5/00; A61B 5/369; G16H 50/20

(52) U.S. Cl. CPC A61B 5/7275; A61B 5/369; A61B 5/486; G16H 50/20

(57) ABSTRACT

A method for predicting prognosis of a patient suffering from a mental disorder, undergoing a neurofeedback (NF) treatment, comprising:
measuring EEG signals from a brain of the patient, before, during and/or after at least one treatment session of the NF treatment;
generating an indicator of activity of at least one specific brain network based on the measured EEG signals;
processing the indicator activity;
predicting in a timed relation with the at least one treatment session, for example before, during and/or after the at least one treatment session, a prognosis of a state of the patient at an end of the NF treatment and/or during the NF treatment, based on the processed activity indicator.

### RELATED APPLICATION/S

This application claims the benefit of priority under 35 USC § 119(e) of U.S. from Provisional Patent Application No. 63/433,025, filed Dec. 16, 2022, the contents of which are incorporated herein by reference in their entirety.

### FIELD AND BACKGROUND OF THE INVENTION

The present invention, in some embodiments thereof, relates to predicting prognosis of a patient in response to a treatment and, more particularly, but not exclusively, to predicting prognosis of a patient in response to NF treatment.

### SUMMARY OF THE INVENTION

Some examples of some embodiments of the invention are listed below (an embodiment of the invention may include features from more than one example and/or fewer than all features of an example):

Example 1. A method for predicting prognosis of a patient suffering from a mental disorder, undergoing a neurofeedback (NF) treatment, comprising:
measuring EEG signals from a brain of said patient, before, during and/or after at least one treatment session of said NF treatment;
generating an indicator of activity of at least one specific brain network based on said measured EEG signals;
processing said indicator activity;
predicting in a timed relation with said at least one treatment session, a prognosis of a state of said patient at an end of said NF treatment and/or during said NF treatment, based on said processed activity indicator.

Example 2. A method according to example 1, wherein said predicting prognosis comprises predicting an improvement in said patient state at an end of at least one session of said NF treatment.

Example 3. A method according to any one of the previous examples, wherein said predicting prognosis comprises predicting an improvement in said patient state at least one week from completing said NF treatment.

Example 4. A method according to any one of the previous examples, wherein said predicting prognosis comprises predicting improvement in one or more clinical assessment exams that is used to measure a clinical state of patients suffering from said mental disorder.

Example 5. A method according to example 4, wherein said predicting improvement comprises predicting an ability of said patient to achieve at least one of, a target score of said one or more clinical assessment exams, a target range of scores of said one or more clinical assessment exams.

Example 6. A method according to any one of examples 4 or 5, wherein said predicting improvement comprises predicting an ability of said patient to reach a target reduction in a score of said one or more clinical assessment exams compared to a reference score or a base line score, when performing said one or more clinical assessment exams.

Example 7. A method according to any one of examples 4 to 6, wherein said mental disorder comprises a stress disorder, and wherein said predicting improvement comprises predicting an ability of said subject to reach a reduction of at least 3 points in a Clinician-Administered post-traumatic stress disorder (PTSD) Scale for DSM-5 (CAPS-5), compared to a reference or a baseline score of said CAPS-5, and/or a reduction of at least 3 points in a PTSD Checklist for DSM-5 (PCL-5), compared to a reference or a baseline score of said PCL-5.

Example 8. A method according to example 7, wherein said stress disorder comprises post-traumatic stress disorder (PTSD).

Example 9. A method according to any one of examples 7 or 8, wherein said NF treatment comprises training the patient to self-control an activity of a limbic system network or activity of an amygdala, and providing a feedback signal to said patient during said training with information about said activity or changes thereof, based on said measured EEG signals.

Example 10. A method according to any one of examples 4 to 6, wherein said mental disorder comprises depression or major depressive disorder (MDD), and wherein said predicting improvement comprises predicting an ability of said subject to reach a reduction of at least 3 points in a Hamilton Depression Rating Scale (HDRS), compared to a reference or a bassline score of said HDRS, and/or a reduction of at least 4 points in a Snaith-Hamilton Pleasure Scale (SHAPS) compared to a reference or a bassline score of said SHAPS, and/or a reduction of at least 6 points in a Montgomery-Åsberg Depression Rating Scale (MADRS) compared to a reference or a bassline score of said MADRS.

Example 11. A method according to any one of examples 4 to 6, wherein said mental disorder comprises Anhedonia, and wherein said predicting improvement comprises predicting an ability of said subject to reach a reduction of at least 5 points in a Snaith-Hamilton Pleasure Scale (SHAPS) compared to a reference or a bassline score of said SHAPS.

Example 12. A method according to any one of the previous examples, wherein said predicting comprises predicting a success of said patient in lowering a score of a CAPS-5 assessment questionnaire in at least 12 points or in at least 6 points relative to a baseline score, after one or more treatment sessions or after one or more weeks of said NF treatment.

Example 13. A method according to any one of examples 1 to 11, wherein said predicting comprises predicting a success of said patient in lowering a score of a CAPS-5 assessment questionnaire in at least 12 points or in at least 6 points relative to a baseline score, after 15 treatment sessions or after 8 weeks of said NF treatment.

Example 14. A method according to any one of the previous examples, wherein said predicting comprises predicting a success of said patient in lowering a score of a CAPS-5 assessment questionnaire in at least 12 points or in at least 6 points relative to a baseline score, after at least 1 week from completing said NF treatment.

Example 15. A method according to any one of the previous examples, wherein said predicting comprises predicting a success of said patient in lowering a score of a CAPS-5 assessment questionnaire in at least 12 points or in at least 6 points relative to a baseline score, after 3 months from completing said NF treatment.

Example 16. A method according to any one of the previous examples, wherein said predicting comprises predicting a success of said patient in increasing a score of a cognitive reappraisal assessment scale in at least 1 point relative to a baseline or a reference score, after one or more treatment sessions or after one or more weeks of said NF treatment.

Example 17. A method according to any one of examples 10 to 16, wherein said NF treatment comprises training the patient to self-control an activity of a mesolimbic system network or activity of the reward system brain network that is associated with the ventral striatum, and providing a feedback signal to said patient during said training with information about said activity or changes thereof, based on said measured EEG signals.

Example 18. A method according to any one of the previous examples, wherein said predicting comprises calculating a prognosis score indicating said prognosis of said patient state, based on said processed activity indicator.

Example 19. A method according to example 18, comprising:
automatically modifying at least one parameter of said NF treatment based on said calculated prognosis score.

Example 20. A method according to example 18, comprising:
delivering an indication with instructions to modify, at least one parameter of said NF treatment based on said calculated prognosis score.

Example 21. A method according to any one of examples 19 or 20, wherein said at least one parameter of said NF treatment comprises at least one of, number of treatment sessions, duration of each or at least one treatment session, an interval between treatment sessions, and a feedback delivered to the patient.

Example 22. A method according to any one of examples 18 to 21, comprising delivering an indication to modify at least one additional treatment provided to the patient.

Example 23. A method according to example 22, wherein said at least one additional treatment comprises at least one of, a pharmaceutical treatment and a psychological treatment.

Example 24. A method according to example 20, wherein if said calculated prognosis score indicates inability of said subject to reach a target improvement, then the delivered indication includes instructions to stop said NF treatment or to add at least one NF treatment session to the NF treatment.

Example 25. A method according to example 24, wherein if said calculated prognosis score indicates inability of said subject to reach a target improvement, then the delivered indication includes instructions to start at least one additional treatment to said NF treatment or to modify said at least one additional treatment.

Example 26. A method according to example 25, wherein if said calculated prognosis score indicates inability of said subject to reach a target improvement then the delivered indication includes instructions to start or to modify at least one of, a pharmaceutical treatment, a psychological treatment and/or a psychotherapy treatment.

Example 27. A method according to example 20, wherein if said calculated prognosis score indicates an ability of said subject to reach a target improvement, then the delivered indication includes instructions to proceed with the NF treatment, or to shorten the NF treatment or a time duration of at least one treatment session.

Example 28. A method according to example 27, wherein if said calculated prognosis score indicates an ability of said subject to reach a target improvement then the delivered indication includes instructions to stop or to modify at least one of, a pharmaceutical treatment, a psychological treatment and/or a psychotherapy treatment.

Example 29. A method according to example 28, wherein if said calculated prognosis score indicates an ability of said subject to reach a target improvement then the delivered indication includes instructions to reduce a dosage of at least one drug administered to said subject during said pharmaceutical treatment.

Example 30. A method according to any one of the previous examples, comprising repeating said measuring and said generating during a selected time period before, during and/or after said at least one treatment session, and wherein said processing comprises identifying changes in said generated activity indicator during said selected time period, and wherein said predicting comprises predicting said prognosis based on the identified changes.

Example 31. A method according to example 30, wherein said processing comprises extracting one or more parameters of said generated activity indication, and wherein said predicting comprises predicting said prognosis based on values of said extracted one or more parameters.

Example 32. A method according to example 31, wherein said processing comprises using values of said extracted one or more parameters in a prediction model, wherein said prediction model comprises weights of said one or more extracted parameters, and wherein said predicting comprises predicting said prognosis based on an output of said prediction model.

Example 33. A method according to example 32, wherein said one or more extracted parameters comprise at least two parameters of said generated indication signal, wherein said prediction model is used to calculate a relation between values of said at least two parameters, and wherein said predicting comprises predicting said prognosis based on said calculated relation.

Example 34. A method according to example 32, wherein said prediction model is used to calculate a relation between said one or more extracted parameters and a reference value, and wherein said predicting comprises predicting said prognosis based on said calculated relation.

Example 35. A method according to any one of the previous examples, wherein said method is performed by a device or a system.

Example 36. A system for calculating a prognosis predicting score, comprising:
a memory, wherein said memory stores a signal indicating activity of at least one specific brain network of a subject suffering from at least one symptom of a mental disorder or changes thereof, measured before, during and/or after a neurofeedback (NF) treatment delivered to said subject, and at least one prognosis prediction software;
a control circuitry, wherein said control circuitry is configured to:
process said stored activity indicating signal;
calculate a prognosis predicting score based on said processing results and using said at least one prognosis prediction software, wherein said prognosis predicting score indicates a prognosis of a state of said subject at an end of said NF treatment and/or during said NF treatment.

Example 37. A system according to example 36, wherein said control circuitry is configured to process said stored activity indicating signal by extracting values of predetermined parameters from said stored activity indicating signal, and wherein said calculate comprises calculate said prognosis predicting score by using said extracted values as input information for said at least one prognosis prediction software.

Example 38. A system according to example 37, wherein said control circuitry is configured to process said stored activity indicating signal by dividing said activity signal into epochs within a range between 1 millisecond and 120 seconds and wherein said extracting comprises extracting said values of said activity indicating signal parameters independently for each epoch of said epochs.

Example 39. A system according to any one of examples 36 to 38, wherein said activity indicating signal stored in said memory is derived from EEG signals measured from said subject brain during said NF treatment.

Example 40. A system according to any one of examples 36 to 39, wherein said prognosis prediction score predicts an ability of said subject to achieve a target score in a clinical assessment exam during, at an end and/or following said NF treatment, wherein said target score or indication thereof is stored in said memory.

Example 41. A system according to any one of examples 36 to 39, wherein said prognosis prediction score predicts an ability of said subject to achieve a target change in a score of a clinical assessment exam relative to a baseline value, during, at an end and/or following said NF treatment, wherein at least one of said target change and/or said baseline value is stored in said memory.

Example 42. A system according to any one of examples 40 or 41, wherein said clinical assessment exam comprises at least one of, a Clinician-Administered PTSD Scale for DSM-5 (CAPS-5) assessment scale, a Patient Health Questionnaire-9 (PHQ-9) exam, a Hamilton Depression Rating Scale exam, a Snaith-Hamilton Pleasure Scale (SHAPS) exam, a Montgomery-Asberg Depression Rating Scale (MADRS) exam, a Patient Health Questionnaire (PHQ) exam, a Clinical Global Impression (CGI) exam, an Attention deficit hyperactivity disorder (ADHD) related exam, an Adult ADHD Investigator Symptom Rating Scale (AISRS) exam, an Adult ADHD Self-Report Scale (ASRS) exam, a Test of Variables of Attention (TOVA) exam, or any derivatives thereof.

Example 43. A system according to any one of examples 36 to 42, wherein said control circuitry is configured to generate a prognosis prediction indication of said calculated prognosis predicting score.

Example 44. A system according to example 43, wherein said prognosis prediction indication comprises at least one suggestion to modify at least one parameter of said NF treatment based on said calculated prognosis predicting score, wherein said at least one suggestion is stored in said memory.

Example 45. A system according to example 44, wherein said at least one parameter comprises at least one of, number of treatment sessions, duration of at least one treatment session, and/or time interval between two treatment sessions.

Example 46. A system according to any one of examples 43 to 45, wherein said prognosis prediction indication comprises at least one suggestion to add at least one additional treatment to said subject or to modify at least one parameter of said at least one additional treatment, wherein said at least one suggestion is stored in said memory.

Example 47. A system according to example 46, wherein said at least one suggestion stored in said memory comprises a suggestion to at least one of, start or stop a pharmaceutical treatment, and/or change a dosage and/or an administration regime of at least one drug administered to said subject.

Example 48. A system according to any one of examples 46 or 47, wherein said at least one suggestion stored in said memory comprises a suggestion to start or stop or modify a transcranial magnetic field (TMS) treatment delivered to said subject.

Example 49. A system according to any one of examples 46 to 48, wherein said at least one suggestion stored in said memory comprises a suggestion to start or stop or modify at least one of, a psychological treatment, a cognitive behavioral therapy (CBT) and a psychotherapy.

Example 50. A system according to any one of examples 43 to 49, comprising: a communication circuitry configured to communicate with at least one remote device; wherein said control circuitry is configured to signal said communication circuitry to deliver said prognosis prediction indication to said remote device.

Example 51. A system according to example 50, wherein said at least one remote device comprises an expert interface configured to generate and deliver a human detectable indication, wherein said control circuitry is configured to signal said expert interface via said communication circuitry to generate and deliver said human detectable indication based on said prognosis prediction indication.

Example 52. A system according to example 51, wherein said expert interface is configured to receive an input and to deliver said input to said memory via said communication circuitry.

Example 53. A system according to example 52, wherein said input comprises at least one of, personal information of said subject, clinical data of said subject, clinical assessment data, pharmaceutical treatment related data, data regarding said at least one symptom of said mental disorder, data regarding said mental disorder, data regarding at least one clinical assessment exam and/or results of said subject in said at least one clinical assessment exam.

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

As will be appreciated by one skilled in the art, some embodiments of the present invention may be embodied as a system, method or computer program product. Accordingly, some embodiments of the present invention may take the form of an entirely hardware embodiment, an entirely software embodiment (including firmware, resident software, micro-code, etc.) or an embodiment combining software and hardware aspects that may all generally be referred to herein as a “circuit,” “module” or “system.” Furthermore, some embodiments of the present invention may take the form of a computer program product embodied in one or more computer readable medium(s) having computer readable program code embodied thereon. Implementation of the method and/or system of some embodiments of the invention can involve performing and/or completing selected tasks manually, automatically, or a combination thereof. Moreover, according to actual instrumentation and equipment of some embodiments of the method and/or system of the invention, several selected tasks could be implemented by hardware, by software or by firmware and/or by a combination thereof, e.g., using an operating system.

For example, hardware for performing selected tasks according to some embodiments of the invention could be implemented as a chip or a circuit. As software, selected tasks according to some embodiments of the invention could be implemented as a plurality of software instructions being executed by a computer using any suitable operating system. In an exemplary embodiment of the invention, one or more tasks according to some exemplary embodiments of method and/or system as described herein are performed by a data processor, such as a computing platform for executing a plurality of instructions. Optionally, the data processor includes a volatile memory for storing instructions and/or data and/or a non-volatile storage, for example, a magnetic hard-disk and/or removable media, for storing instructions and/or data. Optionally, a network connection is provided as well. A display and/or a user input device such as a keyboard or mouse are optionally provided as well.

Any combination of one or more computer readable medium(s) may be utilized for some embodiments of the invention. The computer readable medium may be a computer readable signal medium or a computer readable storage medium. A computer readable storage medium may be, for example, but not limited to, an electronic, magnetic, optical, electromagnetic, infrared, or semiconductor system, apparatus, or device, or any suitable combination of the foregoing. More specific examples (a non-exhaustive list) of the computer readable storage medium would include the following: an electrical connection having one or more wires, a portable computer diskette, a hard disk, a random access memory (RAM), a read-only memory (ROM), an erasable programmable read-only memory (EPROM or Flash memory), an optical fiber, a portable compact disc read-only memory (CD-ROM), an optical storage device, a magnetic storage device, or any suitable combination of the foregoing. In the context of this document, a computer readable storage medium may be any tangible medium that can contain, or store a program for use by or in connection with an instruction execution system, apparatus, or device.

A computer readable signal medium may include a propagated data signal with computer readable program code embodied therein, for example, in baseband or as part of a carrier wave. Such a propagated signal may take any of a variety of forms, including, but not limited to, electromagnetic, optical, or any suitable combination thereof. A computer readable signal medium may be any computer readable medium that is not a computer readable storage medium and that can communicate, propagate, or transport a program for use by or in connection with an instruction execution system, apparatus, or device.

Program code embodied on a computer readable medium and/or data used thereby may be transmitted using any appropriate medium, including but not limited to wireless, wireline, optical fiber cable, RF, etc., or any suitable combination of the foregoing.

Computer program code for carrying out operations for some embodiments of the present invention may be written in any combination of one or more programming languages, including an object oriented programming language such as Java, Smalltalk, C++ or the like and conventional procedural programming languages, such as the “C” programming language or similar programming languages. The program code may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider).

Some embodiments of the present invention may be described below with reference to flowchart illustrations and/or block diagrams of methods, apparatus (systems) and computer program products according to embodiments of the invention. It will be understood that each block of the flowchart illustrations and/or block diagrams, and combinations of blocks in the flowchart illustrations and/or block diagrams, can be implemented by computer program instructions. These computer program instructions may be provided to a processor of a general purpose computer, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks.

These computer program instructions may also be stored in a computer readable medium that can direct a computer, other programmable data processing apparatus, or other devices to function in a particular manner, such that the instructions stored in the computer readable medium produce an article of manufacture including instructions which implement the function/act specified in the flowchart and/or block diagram block or blocks.

The computer program instructions may also be loaded onto a computer, other programmable data processing apparatus, or other devices to cause a series of operational steps to be performed on the computer, other programmable apparatus or other devices to produce a computer implemented process such that the instructions which execute on the computer or other programmable apparatus provide processes for implementing the functions/acts specified in the flowchart and/or block diagram block or blocks.

Some of the methods described herein are generally designed only for use by a computer, and may not be feasible or practical for performing purely manually, by a human expert. A human expert who wanted to manually perform similar tasks, such as predicting prognosis and/or calculating a prediction score, might be expected to use completely different methods, e.g., making use of expert knowledge and/or the pattern recognition capabilities of the human brain, which would be vastly more efficient than manually going through the steps of the methods described herein.

### BRIEF DESCRIPTION OF THE SEVERAL VIEWS OF THE DRAWINGS

Some embodiments of the invention are herein described, by way of example only, with reference to the accompanying drawings. With specific reference now to the drawings in detail, it is stressed that the particulars shown are by way of example and for purposes of illustrative discussion of embodiments of the invention. In this regard, the description taken with the drawings makes apparent to those skilled in the art how embodiments of the invention may be practiced.

In the drawings:

FIG. 1A is a general flow chart of a process for predicting an improvement of a patient following a treatment, for example a neurofeedback treatment, according to some exemplary embodiments of the invention;

FIG. 1B is an illustration showing flow of a NF treatment which includes improvement prediction points, according to some exemplary embodiments of the invention;

FIG. 1C is a graph showing changes in a signal indicating activity of at least one brain network, and changes in an improvement predicting score derived from the activity indicating signal, during and following a NF treatment, according to some exemplary embodiments of the invention;

FIG. 2 is a detailed flow chart of a process for predicting an improvement of a patient during and/or following a NF treatment, according to some exemplary embodiments of the invention;

FIG. 3A is a block diagram of a system for delivery of NF treatment and for predicting patient improvement, optionally online during the delivery of a NF treatment session, according to some exemplary embodiments of the invention;

FIG. 3B is a block diagram of a system for delivery of NF treatment in communication with a remote device used for predicting patient improvement, optionally offline, according to some exemplary embodiments of the invention;

FIG. 4 is a detailed flow chart of a process for calculating a patient improvement predicting score as performed by a system, for example one or both of the systems described in FIGS. 3A and 3B, according to some exemplary embodiments of the invention;

FIG. 5A is a flow chart of a process for processing a brain region activity indicator for calculating an improvement prediction score, according to some exemplary embodiments of the invention;

FIG. 5B is a flow chart of a NF treatment session, according to some exemplary embodiments of the invention;

FIG. 6 is a flow chart describing a process for generating a prediction model, according to some exemplary embodiments of the invention;

FIG. 7 is a heat map showing an example of model accuracy scores;

FIG. 8 is a graph showing an example of changes in a prediction score in patients that did not showed improvement;

FIG. 9 is a graph showing an example of changes in a prediction score in patients that showed improvement; and

FIG. 10 is a table showing an example of changes in a prediction score in each treatment session in three different patients.

### DESCRIPTION OF SPECIFIC EMBODIMENTS OF THE INVENTION

The present invention, in some embodiments thereof, relates to predicting improvement of a patient state in response to treatment and, more particularly, but not exclusively, to predicting improvement of a patient state in response to NF treatment.

A broad aspect of some embodiments relates to predicting prognosis of a subject suffering from at least one mental disorder symptom, for example a subject receiving at least one treatment for treating said mental disorder symptom. In some embodiments, the prognosis prediction comprises predicting a progress of a subject in a treatment process, for example a NF treatment or a combined treatment which includes the NF treatment and at least one additional treatment, for example a pharmaceutical treatment and/or a psychological treatment. In some embodiments, the progress of the subject is predicted based on signals, for example EEG signals, measured from the subject brain indicating activity or changes in activity of at least one specific neural network, optionally associated with a specific brain region. In some embodiments, the measured EEG signals indicate an activity of at least one specific brain region, for example a brain region associated with the specific brain network. Optionally, the EEG signals indicate activity of at least one specific brain region or a plurality of brain regions, for example a plurality of brain regions associated with at least one specific brain network. In some embodiments, the at least one specific brain network comprises a limbic system brain network and the one or more specific brain regions comprise at least one of, an amygdala, a thalamus, hypothalamus, and the hippocampus. In some other embodiments, the at least one specific brain network comprises a reward system brain network and the one or more specific brain regions comprise at least on of, a ventral striatum, ventral pallidum, anterior cingulate cortex, and orbital prefrontal cortex. Alternatively or additionally, the at least one specific brain network comprises a response inhibition brain network and the one or more specific brain regions comprise at least on of, a right inferior frontal gyrus and the right anterior insula.

In some embodiments, the signals are measured in a timed relation with the treatment, for example before, during and/or after, the treatment. In some embodiments, predicting progress of a subject comprises predicting improvement of the subject following one or more treatment sessions of the treatment, predicting progress of a subject towards a desired, for example a target, goal of the treatment, or lack in improvement or progress. Alternatively or additionally, predicting progress of a subject comprises predicting progress relative to a previous state, relative previously measured values, relative to a reference state or a value, and/or relative to a baseline state or value

An aspect of some embodiments relates to predicting prognosis of a subject, for example improvement or decline of a subject undergoing a treatment, for example a neurofeedback (NF) treatment, during the treatment. In some embodiments, the improvement is predicted based on signals measured from the subject brain, for example EEG signals, indicating activity or change in activity of at least one brain network, for example a network associated with the at least one specific brain region. As used herein, predicting improvement of a subject means predicting an improvement or lack of improvement of at least one of, a subject state, a subject mental state, symptom of a mental state, an ability of the subject to comply with the treatment, and an ability of the subject to reach a desired target of the treatment, for example a desired outcome of clinical assessment of the subject state following the treatment.

According to some embodiments,, the subject is a patient diagnosed with or is suspected to have a mental disorder or is predicted to have a mental disorder in the future, for example a stress disorder, depression, Post-traumatic stress disorder (PTSD), Major depressive disorder (MDD), Bipolar disorder (BPD), Attention deficit hyperactivity disorder (ADHD), Obsessive compulsive disorder (OCD), Substance use disorder (SUD), or any other disorder affecting a mental state, for example as described in The Diagnostic and Statistical Manual of Mental Disorders (DSM), for example in the fifth edition of the DSM (DSM-V).

According to some exemplary embodiments, the prognosis, improvement or decline prediction is performed with an accuracy of at least 50%, for example with an accuracy of at least 60%, of at least 70%, of at least 80%, of at least 90%, or any intermediate, smaller or larger percentage value/

According to some embodiments, the overall treatment or at least one parameter of the treatment is modified based on the predicted improvement. In some embodiments, the at least one parameter comprises at least one of, number of treatment sessions of a treatment, interval between two consecutive treatment sessions, length of a treatment session, number and/or length of training blocks in each treatment session, and/or behavior of a stimuli delivered to the patient during the treatment (e.g. noise levels, speed, thresholds). In some embodiments, if the predicted improvement shows no or low predicted improvement in one or more consecutive treatment sessions or in the overall treatment, the treatment is stopped. Alternatively, if the predicted improvement shows no or low predicted improvement in one or more consecutive treatment sessions, one or more additional treatment sessions are added and/or modified. Alternatively or additionally, if the predicted improvement shows no or low predicted improvement in one or more consecutive treatment sessions, the treatment, for example NF treatment, is combined with additional treatment, for example a drug treatment, and/or the adjunct treatment (e.g. pharma) is modified.

According to some embodiments, if the predicted improvement shows an improvement of the subject following one or more treatment sessions, the treatment is shortened. Alternatively, if the predicted improvement shows an improvement of the subject following one or more treatment sessions, a baseline level of a challenge or a stimulus presented to the subject in at least one additional treatment session is increased.

According to some embodiments, a prognosis score is generated based on signals recorded from a subject brain, for example EEG signals. In some embodiments, the EEG signals are processed and the prognosis score is calculated using the processed signals and one or more prognosis predicting software. In some embodiments, a system calculating the prognosis score generates an indication, for example a human detectable indication based on the prognosis score. Optionally, the indication is generated by at least one of, an expert interface configured to generate and deliver an indication to an expert, for example a physician, a technician or any person trained to monitor and/or to control the NF treatment and/or the prediction process, and/or a remote device in communication with the system. Optionally, the remote device comprises the expert interface.

According to some embodiments, if the calculated prognosis score indicates that the subject is predicted to reach a desired target following or during a NF treatment, then an indication is generated by the system. In some embodiments, a desired target comprises at least one of, desired score in a clinical assessment exam, a desired change in a score of the clinical assessment exam compared to a reference value or a baseline score and/or a desired clinical or a behavioral outcome of the NF treatment. In some embodiments, if the calculated prognosis score indicates that the subject is predicted to reach a desired target following or during the NF treatment, the generated indication comprises instruction or suggestions, to modify at least one parameter of the NF treatment and/or to modify at least one parameter of an additional treatment delivered to the subject with the NF treatment. In some embodiments, if the calculated prognosis score indicates that the subject is predicted to reach a desired target following or during the NF treatment, the generated indication comprises instructions to at least one of, shorten a NF treatment, shorten a NF treatment session, prolong an interval duration between treatment sessions, stop a delivery of an additional treatment, change dosage of a drug administered to the subject in the additional treatment, change frequency of other therapy modalities such as pharmacotherapy or transcranial magnetic stimulation (TMS).

According to some embodiments, if the calculated prognosis score indicates inability of the subject to reach a desired target following or during a NF treatment, then the generated indication comprises instructions to at least one of, stop the NF treatment or to add at least one additional NF treatment session to the NF treatment, start at least one additional treatment, in addition to said NF treatment, start or modify at least one of, a pharmaceutical treatment, a psychological treatment, a Cognitive Behavioral Therapy (CBT) treatment and/or a psychotherapy treatment and/or TMS treatment.

According to some embodiments, the NF treatment comprises one or more NF sessions configured to teach a subject self-control, for example self-regulation, of activity of the at least one specific brain network. In some embodiments, the NF treatment teaches the subject self-control the activity of the at least one specific brain network, for example by instructing the subject to self-apply at least one cognitive strategy, for example a cognitive task, and provide a subject an indication regarding the activity or change in the activity of the at least one specific brain network before, during and/or following the self-application of the at least one cognitive strategy. In some embodiments, the indication provided to the subject is generated based on electrical signals measured form the subject brain, for example EEG signals, that are processed using a signature, for example an EEG fingerprint (EFP), to identify EEG signals or parameters thereof in the measured EEG signals that are indicative of the activity or changes thereof of the at least one specific brain network. In some embodiments, the measured EEG signals are processed using the EFP, for example as described in WO2012104853A2, incorporated herein as a reference in its entirety.

According to some embodiments, the cognitive strategy, for example the cognitive task comprises a cognitive or a mental exercise performed by a subject.

According to some embodiments, the improvement of the subject is predicted based only on signals measured from the subject brain during the treatment, for example before, during and/or after at least one treatment session of the treatment. Alternatively, a potential improvement of the subject following a NF treatment is predicted prior to starting the treatment, for example based on EEG signals measured from the subject before starting the NF treatment and/or based on subject diagnosis and/or demographics.

According to some embodiments, the prognosis, for example improvement of the subject is predicted without a need to use any subjective tools, for example questionnaires, an expert interview of the subject and/or observation of the subject by an expert.

According to some exemplary embodiments, the prognosis, for example an improvement of the subject is predicted based on measurements of physiological parameters, for example EEG signals measured between treatment sessions, blood pressure measurements, heart rate measurements and/or any other measurement of a physiological parameter. In some embodiments, the physiological parameters are measured at the subject home. Optionally, an existing prediction of prognosis is updated based on the measured physiological parameters.

According to some embodiments, predicting a prognosis includes predicting a success of a trainee receiving or planned to receive a NF training, also termed herein as NF treatment, in lowering or increasing a score of a clinical assessment questionnaire which indicates improvement of a state, for example a mental and/or a cognitive state of the trainee.

In some embodiments, predicting a prognosis includes predicting a success of the trainee to lower a score of a CAPS-5 assessment questionnaire in at least one point, for example in at least 12 points or in at least 6 points, relative to a baseline score, after one or more training session, for example treatment sessions or after one or more weeks of the NF training.

In some embodiments, predicting a prognosis includes predicting a success of the trainee to lower a score of a PTSD Checklist for DSM-5 (PCL-5) assessment questionnaire in at least one point, for example in at least 3 points, relative to a baseline score, after one or more training session or after one or more weeks of the NF training. In some embodiments, predicting a prognosis includes predicting a success of the trainee to lower a score of a PTSD Checklist for DSM-5 (PCL-5) assessment questionnaire in at least one point after completing the NF training, for example after least 1 week from completion of the NF training.

In some embodiments, predicting a prognosis includes predicting a success of the trainee to lower a score of a Hamilton Depression Rating Scale (HDRS) in at least one point, for example in at least 3 points, relative to a baseline score, after one or more training session or after one or more weeks of the NF training.

In some embodiments, predicting a prognosis includes predicting a success of the trainee to lower a score of a Hamilton Depression Rating Scale (HDRS) in at least one point, for example in at least 3 points after completing the NF training, for example after at least 1 week from completion of the NF training.

In some embodiments, predicting a prognosis includes predicting a success of the trainee to lower a score of a Snaith-Hamilton Pleasure Scale (SHAPS) in at least one point, for example in at least 4 points, relative to a baseline score, after one or more training session or after one or more weeks of the NF training.

In some embodiments, predicting a prognosis includes predicting a success of the trainee to lower a score of a Snaith-Hamilton Pleasure Scale (SHAPS) in at least one point, for example in at least 4 points, after completing the NF training, for example after at least 1 week from completion of the NF training.

In some embodiments, predicting a prognosis includes predicting a success of the trainee to lower a score of a Montgomery-Åsberg Depression Rating Scale (MADRS) in at least one point, for example in at least 6 points, relative to a baseline score, after one or more training session or after one or more weeks of the NF training.

In some embodiments, predicting a prognosis includes predicting a success of the trainee to lower a score of a Montgomery-Åsberg Depression Rating Scale (MADRS) in at least one point, for example in at least 6 points, after completing the NF training, for example after at least 1 week from completion of the NF training.

In some embodiments, predicting a prognosis includes predicting a success of the trainee to lower a score of a Patient Health Questionnaire-9 (PHQ-9) exam in at least one point, for example in at least 6 points, relative to a baseline score, after one or more training session or after one or more weeks of the NF training.

In some embodiments, predicting a prognosis includes predicting a success of the trainee to lower a score of a Patient Health Questionnaire-9 (PHQ-9) exam in at least one point, for example in at least 6 points, after completing the NF training, for example after at least 1 week from completion of the NF training.

In some embodiments, predicting a prognosis includes predicting a success of the trainee to change a score of an Emotion Regulation Questionnaire (ERQ) or components thereof, in at least one point. For example, in some embodiments, predicting a prognosis comprises predicting an increase in at least 1 point of a cognitive reappraisal assessment scale, for example in a cognitive reappraisal facet of the ERQ, relative to a baseline or a reference score, after one or more training session or after one or more weeks of the NF training.

In some embodiments, predicting a prognosis includes predicting a success of the trainee to increase in at least 1 point a score of a cognitive reappraisal assessment scale, for example a cognitive reappraisal facet of the ERQ, after completing the NF training, for example after at least 1 week from completion of the NF training.

A potential advantage of using signals measured from the subject brain may be that the signals allow to have an objective tool for predicting progression, for example improvement, of the subject during or following a treatment, which allows a more accurate and non-biased evaluation and/or prediction.

As used throughout the application, the term “specific brain network” is optionally interchangeable with the term “specific brain region”.

Before explaining at least one embodiment of the invention in detail, it is to be understood that the invention is not necessarily limited in its application to the details of construction and the arrangement of the components and/or methods set forth in the following description and/or illustrated in the drawings and/or the Examples. The invention is capable of other embodiments or of being practiced or carried out in various ways.

### Exemplary General Patient Improvement Prediction

According to some exemplary embodiments, a patient suffering from one or more symptoms of a mental disorder for example a mental disease, undergoes a prediction process which is configured to predict improvement in the one or more of the symptoms after a specific time period and/or following a specific treatment. Alternatively or additionally, the prediction process is configured to predict improvement in a cognitive state of the patient after a specific time period and/or following a specific treatment. Alternatively or additionally, the prediction process is configured to predict the ability of the patient to achieve a target improvement, for example a target clinical improvement, following a specific treatment. In some embodiments, the target clinical improvement is a target clinical improvement as measured by a cognitive scale used to measure improvement of a patient state for a specific mental disorder, for example a stress disorder, a post-traumatic stress disorder scale or a depression scale.

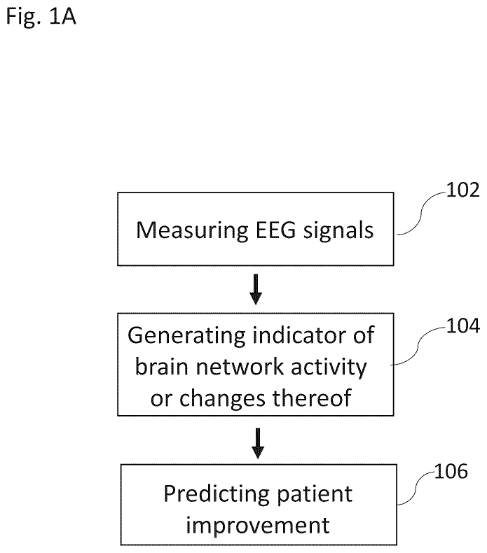

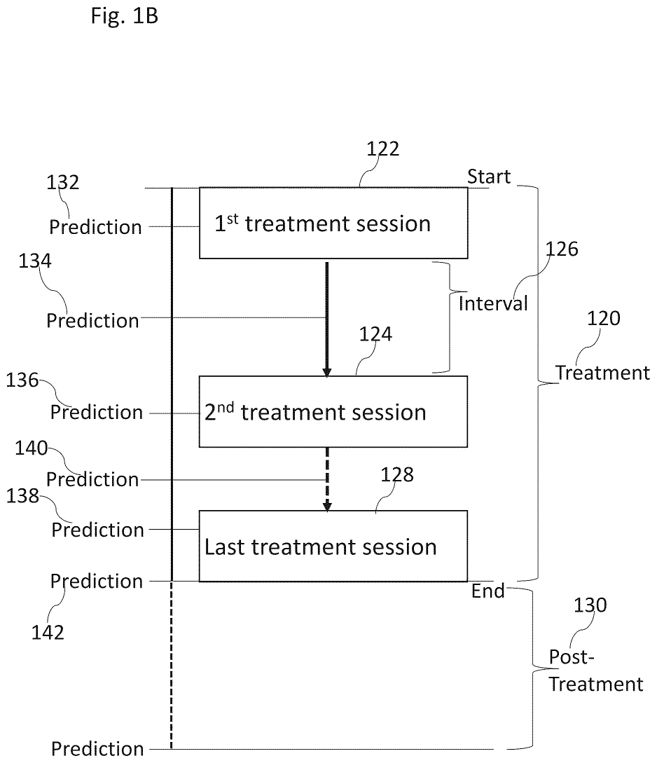

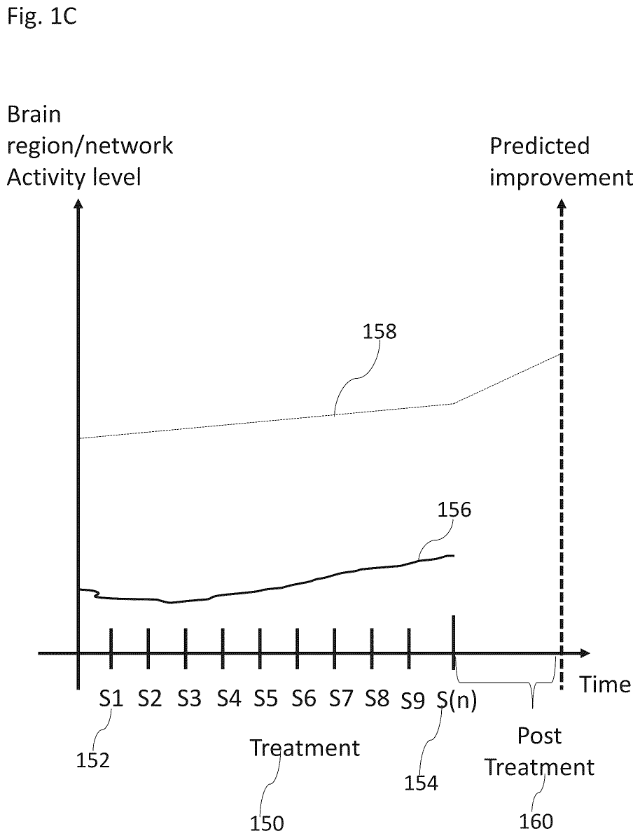

Reference is now made to FIG. 1A, depicting a general process for predicting patient improvement, according to some exemplary embodiments of the invention.

According to some exemplary embodiments, signals from the brain, for example EEG signals, are measured at block 102. In some embodiments, the signals are electrical signals measured by one or more electrodes positioned on a head of a subject, for example by one or more electrodes positioned on a scalp of the subject. In some embodiments, the signals are measured by one or more electrode s positioned on the scalp of the subject, for example by 2 to 6 electrodes, by 3 to 10 electrodes, by 3 to 22 electrodes, or any intermediate, smaller or larger number of electrodes.

According to some exemplary embodiments, the signals are measured in a timed relationship with a treatment session, for example before, during and/or after a treatment session. In some embodiments, the treatment session is a treatment session of a NF treatment which comprises one or more treatment sessions.

According to some exemplary embodiments, the signals are measured from a brain of a subject suffering from, or diagnosed with a mental disorder. In some embodiments, the NF treatment is provided to the subject in order to reduce one or more symptoms associated with the mental disorder and/or to prevent an appearance of the one or more symptoms. In some embodiments the subject is a healthy individual wanting to build resilience to stress or improve at least one of, wellness, concentration, memory.

According to some exemplary embodiments, an indicator of activity or changes in the activity of at least one specific brain network, is generated at block 104. In some embodiments, the indicator is generated using the EEG signals measured at block 102. In some embodiments, the indicator is generated by processing the measured EEG signals with a predetermined signature, for example a predetermined electrical finger print (EFP), of the at least one specific brain network. Optionally or alternatively, the indicator is an indicator of activity or changes thereof of a neural network, for example a neural network associated with the at least one specific brain region, and the EFP is an EFP of the neural network.

According to some exemplary embodiments, the at least one specific brain network is associated with activity in subcortical brain structures, for example limbic structures or brain regions. In some embodiments, these brain regions comprise the amygdala and/or the limbic system.

According to some exemplary embodiments, patient improvement is predicted at block 106. In some embodiments, an improvement of the patient during and/or following the treatment is predicted at block 106. In some embodiments, patient improvement is predicted based on one or more parameters of the generated indicator. Additionally or optionally, the patient improvement is predicted based on one or more parameters of at least one of, the treatment, the patient clinical state, patient history, patient medical history, patient drug regime, and patient personal status, for example age and/or gender.

According to some exemplary embodiments, predicting patient improvement at block 106 comprises predicting that the patient will reach a desired target, for example a desired clinical target or a desired change relative to a reference or baseline, at the end of the treatment, following the treatment, and/or during the treatment. In some embodiments, a desired target comprises a target score in at least one measurement scale measuring one or more symptoms of a disease or a disorder, for example a mental disorder. Alternatively or additionally, a desired target comprises a target score in at least one measurement scale measuring a cognitive state of the patient. Alternatively or additionally, a desired target comprises a target score in at least one measurement scale measuring a potential of the patient to develop one or more symptoms of the disease or disorder, and/or measuring a future cognitive state of the patient.

### Exemplary Prediction of Treatment Outcome

According to some exemplary embodiments, a prediction of a treatment outcome, for example improvement of a patient state or lack thereof, is performed during the treatment. In some embodiments, the prediction is performed during or after a treatment session of the treatment, for example during and/or after the first treatment session or any other treatment session.

Reference is now made to FIG. 1B, depicting prediction of a treatment outcome, according to some exemplary embodiments of the invention.

According to some exemplary embodiments, a treatment 120, for example a NF treatment, comprises one or more separated treatment sessions, for example a first treatment session 122 and a second treatment session 124. In some embodiments, the treatment 120 comprises a plurality of treatment sessions, for example up to 5 treatment sessions, up to 10 treatment sessions, up to 15 treatment sessions, up to 20 treatment sessions, up to 25 treatment sessions, up to 30 treatment sessions or any intermediate, smaller or larger number of treatment sessions. In some embodiments, the treatment sessions were spaced apart by an interval 126, for example a fixed or a varying period of time between consecutive treatment sessions.

According to some exemplary embodiments, the treatment ends with a last treatment session 128, followed by a post treatment period 130. In some embodiments, during each treatment session, for example a NF treatment session, the patient receives a feedback signal indicating activity or changes thereof, of at least one specific brain network. In some embodiments, the specific brain network comprises at least one of, limbic system, mesolimbic system, a network of cognitive or executive control, a network of a positive valence system, a network of a negative valence system. In some embodiments, the feedback signal is generated based on signals measured from the subject brain during the treatment session, for example EEG signals. In some embodiments, the feedback signal is delivered to the patient while the patient is trained to self-control the activity of the at least one specific brain network, for example by performing at least one cognitive task.

According to some exemplary embodiments, the training session is performed in a clinic and/or at a home of the patient using a device that is configured to measure EEG signals and provide a feedback signal to the patient. In some embodiments, during the interval period 126, the patient is optionally engaged in daily activities, and is not receiving any feedback signals regarding the activity of the at least one specific brain network which is based on EEG measurements. Optionally, during the interval period 126, the patient performs the at least one cognitive task practiced during the training session, but without receiving an EEG-based feedback signal. In some embodiments, during the post treatment period which starts when the last treatment session 128 ends, the patient is optionally engaged in daily activities, and is not receiving any feedback signal regarding the activity of the at least one specific brain network which is based on EEG measurements. Optionally, during the post treatment 130, the patient performs the at least one cognitive and/or emotional task practiced during the training session, but without receiving an EEG-based feedback signal.

According to some exemplary embodiments, a prognosis, for example an outcome or progress of the treatment, is predicted during the first treatment session. Alternatively or additionally, the outcome of the treatment is predicted at prediction point 132, at the end of the first treatment session, for example based on EEG signals measured during the treatment session 122 or at the end of the treatment session 122. Alternatively or additionally, a prognosis, for example the outcome of the treatment is predicted at prediction point 134 and 140 during an interval period 126 between two consecutive treatment session, based on EEG signals measured during the interval period 126. In some embodiments, the prognosis is predicted during and/or at an end of each or some treatment sessions, for example at prediction points 136, 138 and 142.

According to some exemplary embodiments, an outcome of the treatment is predicted during and/or following at least one additional treatment session, for example treatment session 124.

According to some exemplary embodiments, prediction of an outcome of the treatment comprises predicting clinical improvement of the patient compared to a reference state, following at least one treatment session, and/or at the end of the treatment, and/or during the post treatment period 130. In some embodiments, predicting clinical improvement comprises predicting improvement in one or more symptoms of a disease or a disorder, for example a mental disorder. In some embodiments, predicting clinical improvement comprises predicting achieving a target change in a desired direction in a score of a test measuring a clinical state, for example a cognitive state, of the patient.

Reference is now made to FIG. 1C depicting prediction of improvement of a subject undergoing a NF treatment based on activity of at least one specific brain network or changes thereof, according to some exemplary embodiments of the invention.

According to some exemplary embodiments, a subject, for example a patient or a subject suffering from at least one symptom of a mental disorder, undergoes a NF treatment 150. In some embodiments, the NF treatment 150 is delivered to the subject during at least one NF treatment session, for example a plurality of treatment sessions, starting with session 1, and ends with session (n) 154, where (n) describes an (n) number of treatment sessions. In some embodiments, the number of treatment sessions is personalized for a subject and/or for a specific clinical state. Optionally, the number of planned treatment sessions is changed during the NF treatment, for example based on at least one of, a performance of the subject during the NF treatment, a clinical state of the subject and/or a prediction of an improvement of the subject during, at the end and/or following the NF treatment 150.

According to some exemplary embodiments, during the NF treatment, the patient practices his ability to self-control an activity of at least one specific brain network, for example a brain network that is associated with activity of amygdala, a brain region of the limbic system. In some embodiments, the at least one specific brain region is associated with deep regions of the brain, for example a sub-cortical brain region. In some embodiments, the subject practices his ability to self-control activity of the at least one specific brain network by performing a cognitive task, optionally while engaging a stimulus, for example a challenge, selected to affect the activation of the at least one specific brain network or to trigger at least one symptom of the mental disorder.

According to some exemplary embodiments, during the NF treatment sessions, the subject receives a feedback signal indicating the activity of the at least aspect of a specific brain network or changes thereof, based on EEG signals measured from the subject brain, for example by one or more electrodes positioned on a head of the subject. In some embodiments, the one or more electrodes are non-invasive electrodes. In some embodiments, the feedback signal, is delivered to the subject online, for example in a time delay smaller than 60 seconds, smaller than 30 seconds, smaller than 15 seconds, smaller than 5 seconds, or any intermediate, smaller or larger time delay from the measurement of the EEG signal.

According to some exemplary embodiments, the feedback signal is derived from an activity indicator signal, calculated from the measured EEG signals, indicating the activity or changes thereof, of the at least one specific brain network, for example activity signal 156. In some embodiments, the activity signal 156 is delivered as the feedback signal to the patient. Alternatively, the activity signal is stored in a memory of the device and is optionally used for predicting an improvement of the patient during and/or following the NF treatment. Optionally, the prediction of the improvement of the patient is based on an improvement predicting score 158 calculated from the activity signal 156. In some embodiments, the improvement prediction score is cumulative, for example an improvement predicting score calculated at an end of a NF treatment session is based on the activity signal calculated during the session and on the improvement prediction score calculated in one or more previous NF treatment sessions. Optionally, calculating a cumulative improvement predicting score increases accuracy and/or stability of the prediction.

According to some exemplary embodiments, the improvement predicting score is calculated at an end and/or during one or more treatment session. In some embodiments, there is a correlation between the activity signal 156 or changes thereof, and the improvement predicting score 158 or changes thereof. In some embodiments, changes in the activity signal 156 precede changes in the improvement predicting score. In some embodiments, the improvement predicting score 158 predicts an improvement in the patient clinical state, for example an improvement in a mental state of the patient, during the NF treatment 150, at an end of the last NF treatment session, for example treatment session S(n), and/or following the NF treatment, at a post treatment 160 time period.

### Exemplary Predicting Prognosis

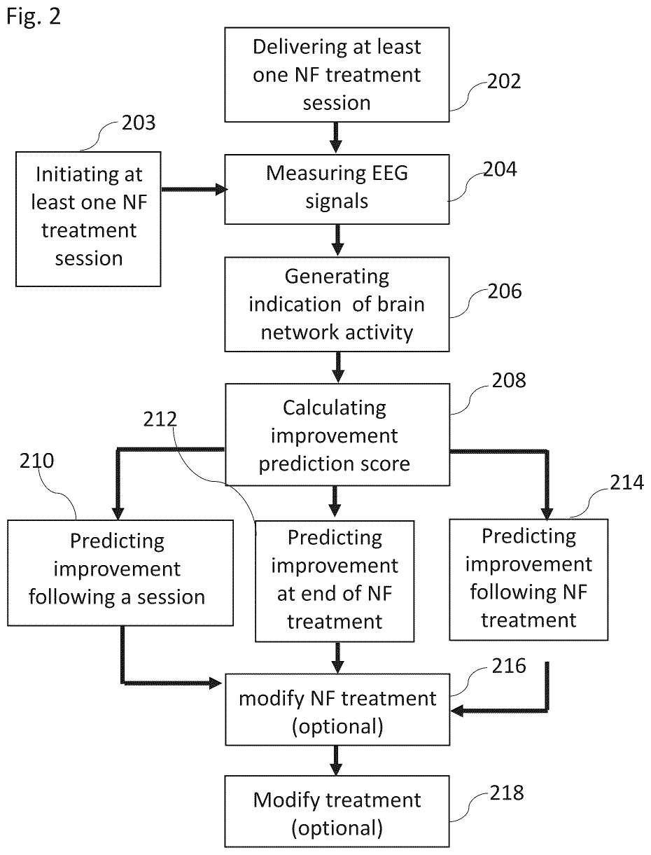

Reference is now made to FIG. 2, depicting a process for predicting prognosis, for example improvement following a NF treatment, according to some exemplary embodiments of the invention.

According to some exemplary embodiments, predicting improvement following the NF treatment comprises predicting improvement following at least one or all planned treatment sessions of a NF treatment, In some embodiments, predicting improvement following the NF treatment comprises predicting improvement of a state, for example a cognitive state, of a subject undergoing the NF treatment. Optionally, predicting improvement of the NF treatment comprises predicting a change in at least one scale measuring at least one parameter of the subject state, following at least one treatment session or all of the treatment sessions of a planned NF treatment.

According to some exemplary embodiments, at least one treatment session of a planned NF treatment is delivered to a subject, for example to a patient, at block 202. In some embodiments, during the treatment session, the patient is trained to control an activation of at least one specific brain network, for example a brain network that is associated with a sub-cortical brain region of the limbic system, by applying a cognitive task. Optionally, the cognitive task is selected to affect the activation of the at least one specific brain network in the patient. In some embodiments, during the treatment session that patient receives on-line, a feedback signal indicating the activity level of the specific brain network based on the measured EEG signals, for example as described in WO2012104853A2, incorporated herein as a reference in its entirety. In some embodiments, an activity level of the at least one specific brain network is determined using at least one signature, for example an electric fingerprint (EFP) of a brain region associated with the specific brain network, for example a signature of the Amygdala described in WO2012104853A2, or a signature of the Ventral Striatum described in WO2021260697A1.

According to some exemplary embodiments, EEG signals are measured at block 204. In some embodiments, the EEG signals are measured before, and/or during and/or following the treatment session. In some embodiments, the EEG signals are measured at block 204 after initiating a NF treatment session at block 203. In some embodiments, the EEG signals are measured by at least one electrode positioned on a head of the subject, for example positioned on a scalp of the subject. In some embodiments, the EEG signals are positioned by a plurality of electrodes positioned on a head of the subject.

According to some exemplary embodiments, at least one indication of the specific brain network activity or changes thereof is generated, at block 206. In some embodiments, the indication is generated based on the measured EEG signals, without additional scan data, for example without simultaneously receiving signals from a functional magnetic resonance imaging (fMRI) device. In some embodiments, the indication is generated and delivered to the patient on-line, for example with a delay of less than 30 seconds, for example less than 20 seconds, less than 10 seconds, less than 5 seconds, less than 3 seconds, less than 1 seconds, or with a delay of any intermediate, shorter or longer time period, from the measurement of the EEG signals at block 204. Optionally, the indication is delivered to the patient as a feedback signal providing feedback to the patient regarding the activation or changes thereof of the specific brain network. Additionally or optionally, the feedback signal is a feedback regarding the ability of the patient to affect the activation of the at least one specific brain network by applying at least one cognitive task, for example as described at block 202.

According to some exemplary embodiments, the indication is generated at block 206 by processing the measured EEG signals with the at least one EFP of the specific brain network. In some embodiments, the processing of the measured EEG signals using the EFP and/or other or an additional calculation comprises using the EFP to isolate a subset of EEG signals from the measured EEG signals, recorded by selected electrodes, having selected frequency bands, that were measured at specific time points. In some embodiments, the EFP includes information regarding the selected electrodes, the selected frequency bands and the selected measurements time points, that indicate activity in the specific brain network. In some embodiments, the EFP is a function that allows to correlate between activation of the specific brain network at a specific time period, for example as measured by fMRI, with the subset of the measured EEG signals, that were optionally measured in a time delay from the fMRI measured activation of the specific brain network.

According to some exemplary embodiments, an improvement prediction score is calculated at block 208. In some embodiments, the improvement prediction score is calculated based on the indication generated at block 206. Alternatively or additionally, the improvement prediction score is calculated based on the EEG signals measured at block 204. In some embodiments, the improvement prediction score is calculated using one or more parameters, for example one or more features of the indication generated at block 206. Alternatively or additionally, the improvement prediction score is generated based on at least one of, one or more parameters of the NF treatment, a clinical state of the patient, a cognitive state of the patient, a drug regime of the patient.

According to some exemplary embodiments, the one or more parameters of the indication comprises at least one of, measures of central tendency, dispersion, entropy of NF sections or the difference in these measures between NF sections, or one or more other features of the indication. In some embodiments, the one or more parameters of the NF treatment comprise at least one of, number of treatment sessions, length of treatment session, interval between consecutive treatment sessions, or any statistical manipulation thereof.

According to some exemplary embodiments, the improvement prediction score is calculated during at least one treatment session, at an end of a treatment session and/or between two consecutive treatment sessions, optionally at a prediction point for example as described in FIG. 1B.

According to some exemplary embodiments, the improvement prediction score is used to predict an improvement of a state of the patient, for example a cognitive state and/or a mental state, following at least one NF treatment session, at block 210. In some embodiments, the improvement prediction score is used to predict an improvement of a state of the patient, following the first treatment session and/or following at least one additional treatment session.

According to some exemplary embodiments, the improvement prediction score is used to predict an improvement of the state of the patient at the end of the NF treatment, for example when finishing the last treatment session of a planned NF treatment, at block 212.

According to some exemplary embodiments, the improvement prediction score is used to predict an improvement of the state of the patient at a time period following the end of the treatment, at block 214. In some embodiments, the improvement prediction score is used to predict an improvement of the state of the patient at the post treatment period 130 describe din FIG. 1B, for example during the post-treatment period 130 or at an end of the post treatment period 130. In some embodiments, the post treatment period lasts for at least 1 week, for example at least 1 month, at least 2 months, at least 3 months, at least 6 months, at least 1 year, following the end of the last treatment session.

According to some exemplary embodiments, if the predicted improvement is not a target improvement, for example a desired improvement, at least one parameter of the NF treatment is modified, at block 216. In some embodiments, if the predicted improvement is lower than the target improvement, the NF treatment or a NF treatment session is stopped, for example if the predicted improvement is lower than at least 80%, for example lower than at least 70%, lower than at least 60%, lower than at least 50%, lower than at least 40%, lower than at least 30% or any intermediate, smaller or larger percentage value, from a target improvement. Alternatively, if the predicted improvement is lower than the target improvement, the number and/or a length of treatment sessions is increased, for example if the predicted improvement is lower than up to 5%, up to 10%, up to 15%, up to 20%, up to 25%, up to 30%, from a target improvement. Optionally, if the predicted improvement is lower than the target improvement, the NF treatment is stopped, and/or an additional treatment, for example a pharmaceutical treatment is added or is modified. Optionally, if the predicted improvement is lower than the target improvement, one or more additional treatment sessions, for example booster treatment sessions are added to the NF treatment.

According to some exemplary embodiments, if the predicted improvement is better than a target improvement, the NF treatment is stopped or shortened, for example the number of planned treatment sessions is reduced. Alternatively, if the predicted improvement is better than a target improvement, the duration of a treatment session is reduced. Alternatively, if the predicted improvement is better than a target improvement, a level and/or complexity of a challenge presented to the patient during the NF treatment session, for example a stimulus, is increased.

According to some exemplary embodiments, the NF treatment, for example at least one parameter of the NF treatment is modified automatically, for example by a device that generates the prediction. Optionally, a device that delivers the NF treatment modifies the NF treatment automatically, optionally, without delivering an indication to the patient and/or expert regarding the modification. Alternatively or additionally, an expert modifies the NF treatment, for example based on one or more indications generated by a device, for example a device that generates the prediction.

According to some exemplary embodiments, if the predicted improvement is not a target improvement, for example a desired improvement, at least one parameter of an overall treatment delivered to the patient is modified, at block 218. In some embodiments, if the predicted improvement is not a target improvement, for example if the predicted improvement is lower than a target improvement, the NF treatment is combined with a psychological treatment and/or with a pharmaceutical treatment using at least one bioactive drug. Alternatively, if the predicted improvement is not a target improvement, for example if the predicted improvement is lower than a target improvement, the NF treatment is replaced with a psychological treatment and/or with a pharmaceutical treatment using at least one bioactive drug.

According to some exemplary embodiments, if the predicted improvement is better than a target improvement, at least one additional treatment, for example a psychological treatment, a cognitive behavioral treatment (CBT), a psychotherapy treatment, a transcranial magnetic stimulation (TMS), and/or a pharmaceutical treatment, delivered to the patient in addition to the NF treatment, is stopped or modified, for example shortened. Alternatively, the at least one additional treatment is started based on the predicted improvement. In some embodiments, if the predicted improvement is better than a target improvement, then an additional pharmaceutical treatment delivered to the subject together with the NF treatment is modified, for example stopped, or a dosing of at least biological agent, for example a drug, is modified, for example reduced.

According to some exemplary embodiments, the at least one additional treatment is modified, started or stopped, based on a predicted prognosis, for example a predicted improvement.

### Exemplary System

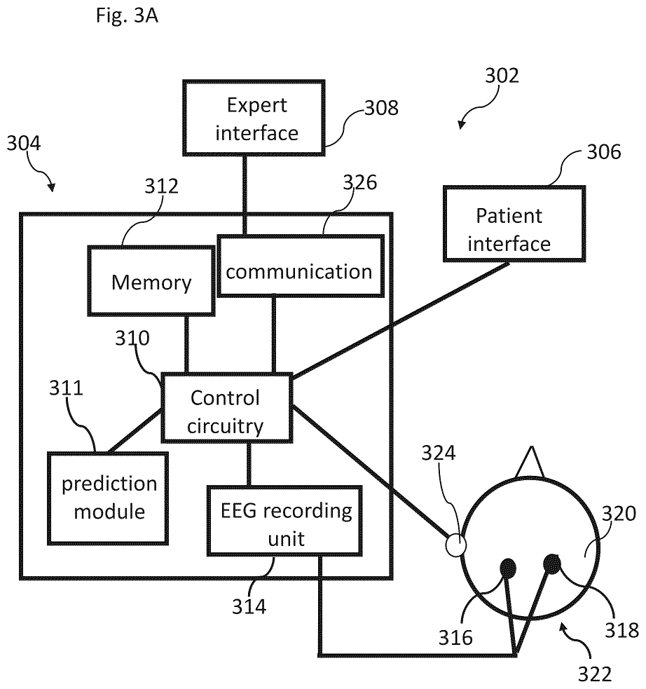

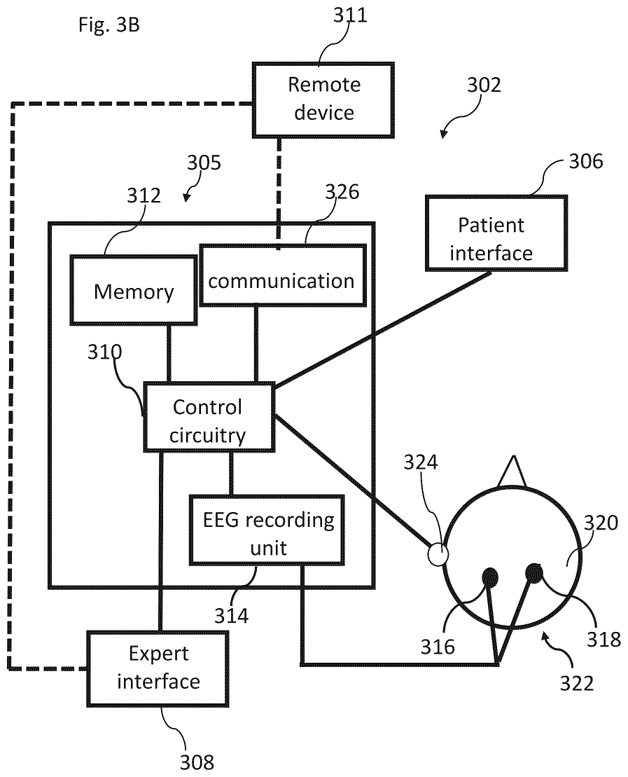

Reference is now made to FIGS. 3A and 3B, depicting a system for delivery of a NF treatment and for prediction of a patient improvement, according to some exemplary embodiments of the invention.

According to some exemplary embodiments, a system 302 comprises a control unit 304, a patient interface 306 and a supervisor interface 308. In some embodiments, the patient interface 306 and the supervisor interface are functionally coupled to a control circuitry 310 of the control unit 304. In some embodiments, the control unit 304 further comprises a memory 312 which stores at least one algorithm and/or a lookup table, to be used when determining an activity level of at least one specific brain network. Alternatively or additionally, the memory 312 comprises at least one EFP, for example an EFP model of the at least one specific brain region, for example the EFP model described in WO2021260697A1, incorporated herein as a reference in its entirety. In other embodiments, the EFP model is stored in the memory 312 of the control unit.

According to some exemplary embodiments, the control unit 304 comprises an EEG recording unit 314 functionally coupled to the control circuitry 310. In some embodiments, the EEG recording unit 314 delivers signals from one or more electrodes positioned on a head of a subject to the control circuitry 310. In some embodiments, the control circuitry 310 is configured to process the received signals and to analyze the processed signals in order to determine an activity level of the at least one specific brain network, and/or a relation between the determined activity and at least one reference value or indication thereof, stored in the memory 312. In some embodiments, the control circuitry 310 determines the activity level or the relation between the activity level and the at least one reference value or indication thereof, using the at least one of an algorithm, a lookup table and/or an EFP stored in the memory 312, for example as described in WO2021260697A1 or in WO2012104853A2, incorporated herein as a reference in their entirety.

According to some exemplary embodiments, the EFP is specific for a brain network, for example a network associated with activity in deeply located brain region, and/or sub-cortical brain region, and/or a brain region of the limbic or mesolimbic system, and/or the amygdala. In some embodiments, the control circuitry uses the EFP to isolate a subset of EEG signals indicating activity of the specific brain network, from the EEG signals received by the EEG recording unit. In some embodiments, the EFP includes information regarding specific electrodes, specific frequency bands and specific recording time points of the subset of EEG signals.

According to some exemplary embodiments, the EEG recording unit is functionally coupled to at least one electrode, for example a plurality of electrodes 316 and 318 positioned on a head 612 of a patient 322. In some embodiments, the plurality of electrodes comprise 2,3,4,5,6,7,8,9,10 or any larger number of electrodes positioned and optionally attached to the head 320 of the patient 322. Optionally, the plurality of electrodes are arranged in an array. In some embodiments, the electrodes are positioned at specific locations on a head of the subject, for example at one or more of the locations Fz, C3, C4, Cz, FCz, P3, Pz and P4 of the extended 10-20 coordinate system. Alternatively, electrodes are positioned in any location or combination of locations of the extended 10-20, 10-10, or any well-established coordinate system. In some embodiments, the control unit 304 is optionally connectable to at least one speaker or earphone 324 configured to deliver an audio signal to the patient 322. In some embodiments, if the system is used for treating a stress disorder, for example PTSD, the electrodes are positioned at locations FCz, and Pz or at Fz, Cz and Pz. In some embodiments, if the system is used for treating depression, for example MDD, the electrode are positioned at locations FCz, Cz, Pz, C3, C4, P3 and P4.

According to some exemplary embodiments, the control unit comprises a communication circuitry 326 configured to receive and/or deliver signals, for example wireless signals to a remote device located outside the supervisor clinic, for example to a remote computer, a remote server or a remote cloud. In some embodiments, the remote device stores the at least one algorithm, lookup table and/or the EFP. In some embodiments, the control unit 304 transmits the electrical signals or processed electrical signals to the remote device via the communication circuitry 326 and receives via the communication circuitry 326 signals which indicate an activity level of the specific brain network and/or a relation between the activity level of the brain network and a reference value indicating a target activation level of the brain network.

Alternatively, the control circuitry 310 generates at least one signal indicating an activity level of the specific brain network and/or a relation between the activity level of the brain network and a reference value indicating a target activation level of the brain network, based on the processing of the received EEG signals using the EFP.

According to some exemplary embodiments, the control circuitry 310 signals the patient interface 306 to deliver a feedback signal, for example to display on a screen a visual signal and/or to provide an audio signal and/or to provide tactile and/or to deliver olfactory signals, indicating the activity of the at least one specific brain network, based on the signals generated by the control circuitry. In some embodiments, the feedback signal continuously changes in response to an activation level of the at least one specific brain network or changes in the activation level relative to a reference value. In some embodiments, the delivered feedback signal is updated every 1 second, 5 seconds, every 10 seconds, every 15 seconds, every 20 seconds, every 25 seconds or any intermediate, smaller or larger value, based on the signals received form the electrodes, for example electrodes 316 and 318, and/or the signals generated by the control circuitry 310 when processing the received EEG signals using the EFP.

According to some exemplary embodiments, the patient interface 306 changes the feedback signal delivered to the patient, for example the visual interface presented to the patient, in a delay of about 1 second, 2 seconds, about 5 seconds, about 10 seconds, about 15 seconds, about 20 seconds, about 25 seconds, or any intermediate, smaller or larger value, relative to the timing in which the electrical signal is received from the electrodes 316 and 318. In some embodiments, the patient interface comprises a display and/or speakers.

According to some exemplary embodiments, the feedback signal, optionally delivered via the patient interface is delivered as a two dimension (2D) visual signal, or as a three dimension (3D) visual signal. In some embodiments, the feedback signal is delivered using virtual reality, augmented reality, with or without at least one of, audio, olfactory and tactile signals.

According to some exemplary embodiments, the control unit 304 comprises a prediction module 311 operationally coupled to the control circuitry 310. In some embodiments, the prediction module 311 is configured to generate an improvement prediction score based on the indication related to the activity of the at least one specific brain region generated by the control circuitry 310. In some embodiments, the prediction module 311 generates the improvement prediction score based on one or more parameters, for example features, of the activity indication.

According to some exemplary embodiments, the control circuitry 310 signals the supervisor interface to deliver visual and/or audio indications to a supervisor of the treatment. In some embodiments, the visual and/or audio indications present at least one of, a progress of the patient during the treatment, activity level of the brain network, changes in the activity level of the brain network, difference between a reference value and an activity level of the brain network, stage of the NF session, actions of the system and indications or cues delivered to the patient. Additionally, the control circuitry signals the user interface to deliver visual and/or audio indications to a supervisor of the treatment with information regarding the improvement prediction score and/or suggestion to modify at least one parameter of the NF treatment, and/or the overall treatment based on the improvement prediction score.

According to some exemplary embodiments, the supervisor interface 308 comprises a remote device, for example a remote computer of the supervisor, coupled to the control unit 304 via the communication circuitry 326.

According to some exemplary embodiments, the control unit 304 transmits at least one of, data collected from the patient, data transmitted or displayed to the supervisor, the improvement prediction score calculated by the prediction module 311, to a remote device, for example to a remote database, a cloud storage or a remote computer, for optionally generating a database. In some embodiments, the database includes information collected from a plurality of systems and/or from a plurality of patients.

According to some exemplary embodiments, for example as shown in FIG. 3B, control unit 305 does not include an integrated prediction module 311. In some embodiments, the prediction module is part of a remote device 311, for example a remote computer, a remote server, and/or a remote cloud storage. In some embodiments, the remote device 311 is in communication with the control unit 305 via the communication circuitry 326. In some embodiments, an activity indication generated by the control circuitry 310 is transmitted to the remote device 311 via the communication circuitry 326. In some embodiments, the remote device 311 calculates an improvement prediction score based on the activity indication received form the communication circuitry 326 and one or more algorithms, look-up tables, formulas, and/or software installed in the memory of the remote device 311.

According to some exemplary embodiments, the remote device delivers an indication with information regarding the calculated improvement prediction score to the expert interface 308. Alternatively, the indication is delivered to the expert interface from the control unit 305 based on signals received from the remote device 311. In some embodiments, the indication delivered to the expert includes one or more suggestions how to modify the NF treatment and/or how to modify the overall treatment delivered to the patient based on the calculated improvement prediction score.

### Exemplary Process for Predicting Patient Improvement

According to some exemplary embodiments, a process for predicting patient improvement is performed by a device or a system that delivers NF treatment to the patient. Alternatively, the prediction process is performed by a separate different device, for example a device that is in communication with the NF treatment device. In some embodiments, the separate device receives signals from the NF device with information or indications regarding the activity of at least one specific brain network. In some embodiments, the separate device uses the received information or indications to calculate a prediction score, and to deliver the prediction score to an expert and/or optionally to the patient. Optionally, the separate device provides instructions and/or suggestions how to modify the NF treatment and/or an overall treatment of the patient, to the expert.

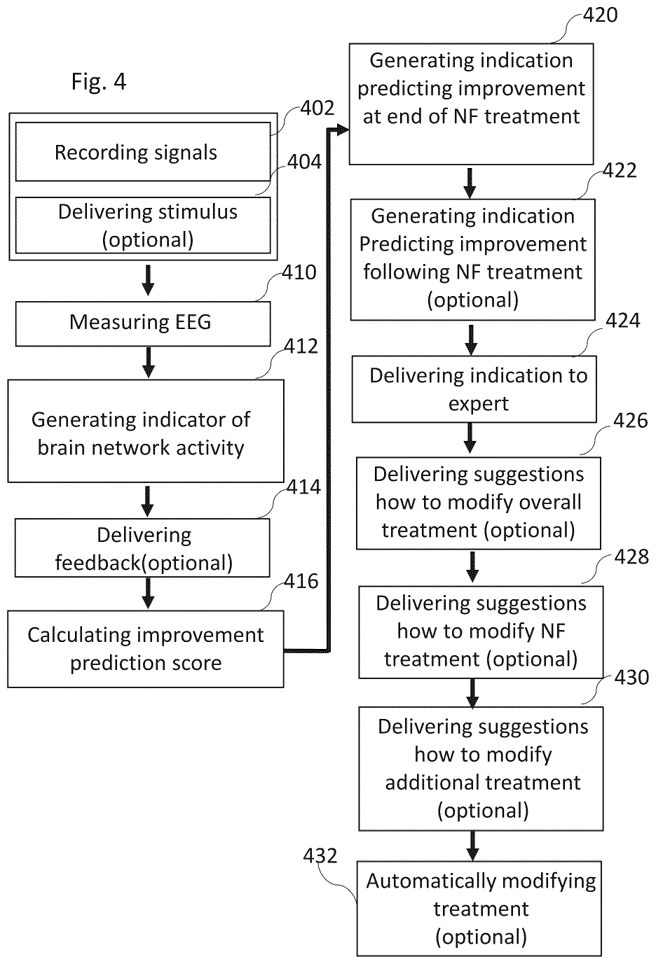

Reference is now made to FIG. 4, depicting a process for predicting patient improvement, as performed by one or more devices, according to some exemplary embodiments of the invention.

According to some exemplary embodiments, signals, for example electrical signals, are recorded from a patient brain, at block 402. In some embodiments, the signals are recorded by at least one electrode, for example electrodes 316 and 318 shown in FIGS. 3A and 3B. In some embodiments, the signals are recorded by at least one electrode, for example a plurality of electrodes, attached to the subject head.

According to some exemplary embodiments, at least one stimulus is optionally delivered to the patient, at block 404. In some embodiments, the stimulus is delivered in a timed relationship with the signals recording, for example before, during and/or after recording the signals at block 402. In some embodiments, the stimulus comprises at least one of, a visual stimulus, an audio stimulus, a tactile stimulus, a sensory stimulus, and an olfactory stimulus. In some embodiments, the stimulus is selected to affect an activation, for example increase activation or reduce activation, of at least one specific brain network or a specific brain region, optionally associated with the brain network.

According to some exemplary embodiments, the stimulus is selected to affect an activation level of the specific brain network, compared to baseline activation of the specific brain network, for example the activation level of the specific brain network when the stimulus is not delivered to the patient. In some embodiments, the stimulus and/or the recorded signals are stored in the memory 312 shown in FIGS. 3A and 3B.

According to some exemplary embodiments, EEG signals are measured from the recorded signals, at block 410. In some embodiments, the EEG signals are measured by the control circuitry 310 shown in FIGS. 3A and 3B, optionally using at least one of, an algorithm, a formula, and a lookup table, stored in the memory 312. In some embodiments, the EEG signals are measured online, for example in less than 30 seconds, less than 20 seconds, less than 10 seconds or any intermediate, shorter or longer time period from the recording of the signals at block 402. Alternatively, the EEG signals are measured in a delay of at least 30 seconds, at least 1 minute, at least 5 minutes, or any intermediate, shorter or longer time period, from the recording of the signals at block 402. In some embodiments, the EEG signals are measured after stopping the signals recording.

According to some exemplary embodiments, an indicator of activity of at least one specific brain network or changes thereof is generated at block 412. In some embodiments, the indicator is generated by the control circuitry 310. In some embodiments, the control circuitry is configured to apply at least one EFP of the specific brain network on the measured EEG signals, in order to generate the activity indicator. In some embodiments, the EFP comprises a model which includes information regarding at least one of, a subset of selected one or more electrodes, selected frequency bands in the measured EEG signals, and specific recording time points, that when applied on the measured EEG signals provide an indication of the activity of the at least one specific brain network. Optionally, the model is used as a filter applied on the measured EEG signals, to identify a subset of EEG signals in the measured EEG signals, that indicate activity or changes thereof of the at least one specific brain network.

According to some exemplary embodiments, the indicator generated at block 412 changes over time according to changes in the measurements of the EEG signals at block 410, and/or according to changes in the signals recorded at block 402. In some embodiments, the indicator is generated, as previously described in WO2012104853A2, incorporated herein as a reference in its entirety.

According to some exemplary embodiments, the EFP, a model of the EFP and/or the generated activity indicator are stored in the memory 312.

According to some exemplary embodiments, a feedback signal is optionally delivered to the patient, at block 414. In some embodiments, the feedback signal comprises the activity indicator generated at block 412. Alternatively, the feedback signal comprises a visual and/or an audio interface which is modified according to the activity indicator generated at block 412 or changes in the activity indicator. In some embodiments, the feedback signal is generated by the control circuitry 310 and is delivered to the patient via the patient interface 306, shown in FIGS. 3A and 3B. In some embodiments, the feedback signal is optionally delivered to the patient, when the EEG signals are measured as part of a NF treatment.

According to some exemplary embodiments, an improvement prediction score is calculated at block 416. In some embodiments, the improvement prediction score is calculated by the control unit 304, for example by the prediction module 311 shown in FIG. 3A. Alternatively, the improvement prediction score is calculated by a remote device in communication with the control unit, for example as shown in FIG. 3B. In some embodiments, the improvement prediction score is calculated by applying a prediction algorithm, for example a prediction model, on the activity indicator generated at block 412. Alternatively, the prediction algorithm is applied on the EEG signals measured at block 410.

According to some exemplary embodiments, the improvement prediction score is calculated prior to initiating a NF treatment, based on signal recorded at block 402 prior to initiating the NF treatment, for example during a baseline or a screening procedure of the NF treatment. Alternatively, the improvement prediction score is calculated during and/or following at least one treatment session of the NF treatment.

According to some exemplary embodiments, an indication regarding suitability of the patient to receive the NF treatment is optionally generated at block 418. In some embodiments, the indication is generated based on EEG signals measured during a baseline and/or a screening session of the NF treatment, prior to initiating the first treatment session.

According to some exemplary embodiments, an indication predicting improvement of a state of the patient at an end of the NF treatment and/or during the NF treatment is generated at block 420. In some embodiments, the indication is generated by the control circuitry 310 or by the remote device 311. In some embodiments, the indication is generated prior to initiating the NF treatment. Alternatively or additionally, the indication is generated during and/or at an end of at least one treatment session of the NF treatment. Alternatively or additionally, the indication is generated at an interval period between two consecutive treatment sessions. In some embodiments, the indication includes information regarding the ability of the patient to reach a desired target value in a scale measuring at least one symptom of a mental disorder or severity of the mental disorder, at the end of the NF treatment. In some embodiments, the indication includes information regarding the ability of the patient to achieve a reduction, for example a target reduction is a score of the scale, at the end of the NF treatment, for example at a beginning of the last NF treatment session, during the last NF treatment session and/or at the end of the last treatment session.

According to some exemplary embodiments, the indication includes information regarding the ability of the patient to achieve a reduction of at least 3 points, for example a reduction of at least 5 points, a reduction of at least 6 points, a reduction of at least 10 points, a reduction of at least 12 points, a reduction of at least 20 points or any intermediate, smaller or larger reduction, in at least one clinical assessment exam. In some embodiments, the clinical assessment exam comprises a post-traumatic stress disorder (PTSD) exam, for example a Clinician-Administered PTSD Scale for DSM-5 (CAPS-5) and/or parameters or domains of the CAPS-5, a PTSD Checklist for DSM-5 (PCL-5), and a Patient Health Questionnaire-9 (PHQ-9).

Alternatively or additionally, the clinical assessment exam comprises a depression related exam, for example the Hamilton Depression Rating Scale or any derivative, the Snaith-Hamilton Pleasure Scale (SHAPS) or any derivative, the Montgomery-Asberg Depression Rating Scale (MADRS) or derivatives, the Patient Health Questionnaire (PHQ) or derivatives, the Clinical Global Impression (CGI) or derivatives. Alternatively or additionally, the clinical assessment exam comprises an Attention deficit hyperactivity disorder (ADHD) related exam, for example the Adult ADHD Investigator Symptom Rating Scale (AISRS) or derivatives, the Adult ADHD Self-Report Scale (ASRS) or derivatives, the Test of Variables of Attention (TOVA), the Patient Health Questionnaire (PHQ), and the CGI.

According to some exemplary embodiments, an indication predicting improvement of a state of the patient at following the NF treatment is generated at block 422. In some embodiments, the indicating is an indicating predicting improvement of the patient up to 1 week, up to 1 month, up to 3 months, up to 6 months, up to a year, or any intermediate, shorter or longer time period from ending the last NF treatment session and/or from one or more NF sessions. In some embodiments, the indication is generated by the control circuitry 310 or by the remote device 311. In some embodiments, the indication is generated prior to initiating the NF treatment. Alternatively or additionally, the indication is generated during and/or at an end of at least one treatment session of the NF treatment. Alternatively or additionally, the indication is generated at an interval period between two consecutive treatment sessions.

According to some exemplary embodiments, the indication includes information regarding the ability of the patient to reach a desired target value in a scale measuring at least one symptom of a mental disorder or severity of the mental disorder, following the NF treatment. In some embodiments, the indication includes information regarding the ability of the patient to achieve a reduction, for example a target reduction is a score of the scale, following the NF treatment, for example up to 1 week, up to 1 month, up to 3 months, up to 6 months, up to a year, or any intermediate, shorter or longer time period from ending the last NF treatment session or from the one or more NF sessions.

According to some exemplary embodiments, the indication includes information regarding the ability of the patient to achieve a reduction of at least 2 points, for example a reduction of at least 5 points, a reduction of at least 6 points, a reduction of at least 10 points, a reduction of at least 12 points, a reduction of at least 20 points or any intermediate, smaller or larger reduction, in the at least one clinical assessment exam.

According to some exemplary embodiments, the indication is delivered to an expert, at block 424. In some embodiments, the control circuitry, for example control circuitry 310, signals the expert interface to deliver the indication to the expert using the expert interface. Alternatively, in case the expert interface is in communication with a remote device, for example as shown in FIG. 3B, the control circuitry 310 signals the remote device 311 using the communication circuitry 326 to deliver the indication via the expert device 308 to the expert.

According to some exemplary embodiments, suggestions or instructions how to modify at least one parameter of an overall treatment provided to the patient, is optionally delivered at block 426. In some embodiments, the suggestions or instructions are part of the indication delivered to the expert at block 424. In some embodiments, the suggestions or instructions, comprise at least one of, providing at least one treatment to the patient optionally in combination, in addition with the NF treatment or as a replacement of the NF treatment, for example a social treatment, a psychological treatment, a psychiatric treatment, a pharmaceutical treatment.

According to some exemplary embodiments, suggestions or instructions on how to modify at least one parameter of the NF treatment provided to the patient, are optionally delivered at block 428. In some embodiments, the suggestions or instructions are part of the indication delivered to the expert at block 424 In some embodiments, the suggestions or instructions, comprise at least one of, stopping the NF treatment, adding one or more additional treatment sessions, changing a length of one or more treatment sessions, changing a time interval between treatment sessions, changing a difficulty or a complexity baseline of a stimulus delivered to the patient during a treatment session.

According to some exemplary embodiments, suggestions or instructions on how to modify at least one additional treatment provided to the subject, for example in addition to the NF treatment, are optionally delivered at block 430. In some embodiments, if the additional treatment comprises a pharmaceutical treatment, the suggestions or instructions include stopping the pharmaceutical treatment, initiating a pharmaceutical treatment and/or modifying a dosage of at least one drug, for example a bioactive compound, administered in the pharmaceutical treatment. In some embodiments, if the additional treatment comprises a psychological treatment, the suggestions or instructions comprise stopping or initiating, or shortening or prolonging the psychological treatment or TMS.

According to some exemplary embodiments, the suggestions or instructions provided to the expert at blocks 426 and/or 428 are stored in a memory of the control unit, for example memory 312, or in a memory of the remote device 311. In some embodiments, the suggestions or instructions are selected and provided to the patient based on the calculated improvement prediction score. Additionally or optionally, the suggestions or instructions are based on at least one of, clinical data of the patient, medical history of the patient, success or failure in previous NF treatment sessions and/or previous NF treatments, and drug regime of the patient. In some embodiments, the suggestions or instructions are selected using at least one of, an algorithm, a formula, and a lookup table, optionally associating an improvement prediction score with a predetermined set of instructions stored in the memory 312 or in the memory of the remote device 311

According to some exemplary embodiments, the system automatically modifies at least one parameter of the treatment, for example the NF treatment, provided to the patient, at block 432. Optionally the system automatically modifies the NF treatment without providing indications, for example instructions or suggestions to the patient or the expert. Alternatively or additionally, an indication that the at least one parameter was modified, is delivered to the patient and/or expert and/or is stored in a memory, for example memory 312 of the device.

### Exemplary Prediction Score Generation

According to some exemplary embodiments, a device, for example a control unit, a computer, a server or a cloud storage and/or processing memory, calculates an improvement prediction score using one or more algorithms, formulas, lookup tables and/or models, stored in a memory of the device. In some embodiments, the prediction score is calculated using a model, for example a model selected for a specific patient, optionally based on at least one of, clinical data, performance of the subject in a NF treatment, medical history, and drug regime. Optionally, a control circuitry of the device calculates the prediction score.

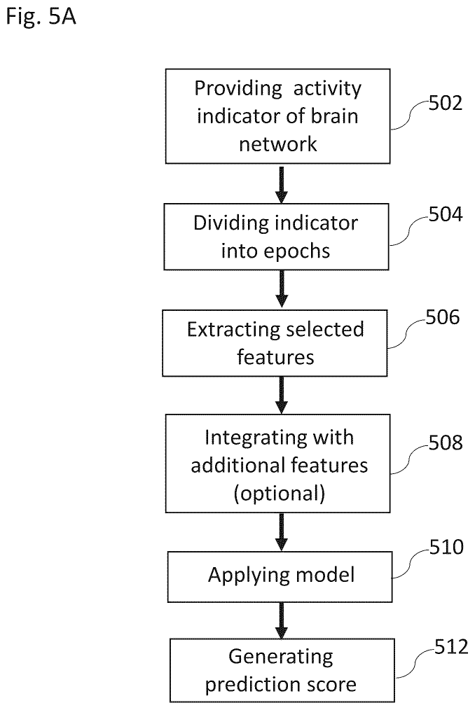

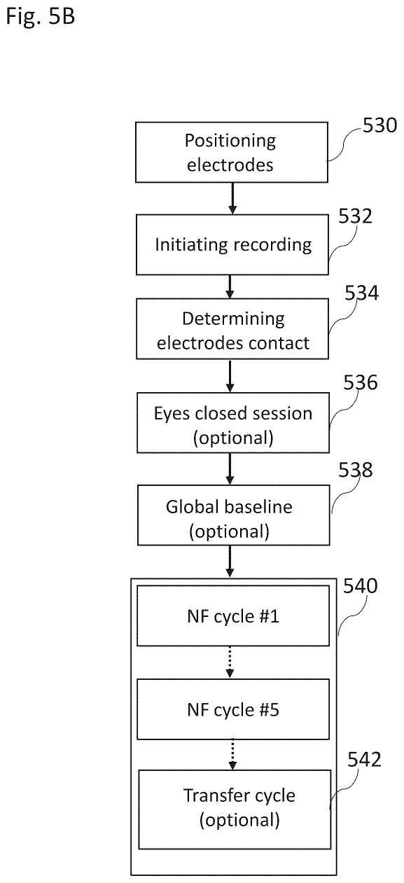

Reference is now made to FIG. 5A, depicting calculation of an improvement prediction score, according to some exemplary embodiments of the invention.

According to some exemplary embodiments, an activity indicator of a brain network is provided at block 502. In some embodiments, the activity indicator is stored in a memory of the device. In some embodiments, the activity indicator is a signal indicating activity or changes thereof of at least one specific brain network. In some embodiments, the activity indicator is generated, for example as described at blocks 104, 206, and/or block 412. In some embodiments, the activity indicator comprises an EFP signal or changes thereof. In some embodiments, the activity indicator is a signal having a time duration of at least 1 millisecond, for example at least 1 second, at least 1 minute, at least 2 minutes, at least 5 minutes, at least 10 minutes, at least 30 minutes, at least 40 minutes, or any intermediate, shorter or longer time period. In some embodiments, the signal indicates an activity of at least one brain network or changes thereof, during a baseline part of a NF treatment session, and/or when a stimulus is presented to the patient during the NF treatment session.

According to some exemplary embodiments, the provided indicator is divided into epochs, at block 504, for example by a control circuitry and/or a prediction module of the device. In some embodiments, the indicator signal provided at block 502 is divided into one or more epochs, each of at least 1 seconds, for example epochs of at least 40 seconds, at least 1 minute, at least 90 seconds, at least 2 minutes, or any intermediate, shorter or longer epochs time. In some embodiments, a length of the epochs is identical. Alternatively, the length of at least some of the epochs varies.

According to some exemplary embodiments, one or more predetermined features are extracted from the indicator signal, at block 506. In some embodiments, the one or more predetermined features are extracted, for example values of the features are extracted, separately for each epoch of the indicator signal. In some embodiments, the features values comprise at least one of, absolute features values and relative features values.

According to some exemplary embodiments, the extracted features are optionally integrated with additional features, at block 508, to form a collection of features. In some embodiments, the additional features comprise Demographic data and/or, EEG data and/or accelerometer measurements, eye-tracking, cardiovascular measurements, etc.

According to some exemplary embodiments, a prediction model, optionally a selected prediction model, is applied on the values of the extracted features, at block 510. In some embodiments, the model is applied on the values of the extracted features and optionally in addition on the values of the integrated additional features, for example on values of the collected features. In some embodiments, the features values are used as an input for the model. Optionally, the model is adjusted for a specific subject or a specific group of subjects, for example a specific patient or group of patients.

According to some exemplary embodiments, the prediction model comprises weights of one or more features, for example parameters. In some embodiments, the model is used to calculate a relation between two or more extracted parameters values. Alternatively or additionally, the model is used to calculate a relation between values of at least one parameter and a reference value.

According to some exemplary embodiments, a prediction score is generated at block 512. In some embodiments, the prediction score is an output of the model, applied at block 510 of the features values. In some embodiments, the prediction score is generated based on the calculated relation between the at least two parameter values or based on the calculated relation between values of at least one parameter and the reference value. Alternatively or additionally, the prediction score is generated based on the different weights of the one or more parameters.

According to some exemplary embodiments, actions described at blocks 504, 506, 508, 510 and 512 are performed by the device, for example by a control circuitry and/or a prediction module of the device, for example by control circuitry 310 and/or prediction module 311.

### Exemplary NF treatment session

According to some exemplary embodiments, a NF treatment for treating PTSD comprises a plurality of treatment sessions. In some embodiments, the treatment sessions are performed during at least 2 consecutive weeks. In some embodiments, the treatment sessions are performed during a total time period of 2 to 20 weeks, for example 2 to 12 consecutive weeks. In some embodiments, the NF treatment comprises at least 5 treatment sessions, for example 10, 12 and 15 treatment sessions, performed during a time period of 5 to 12 weeks. In some embodiments, at least two treatment sessions are performed each week, or in some of the weeks.

Reference is now made to FIG. 5B, depicting a treatment session of a NF treatment for treating subjects diagnosed with PTSD, according to some exemplary embodiments of the invention.

According to some exemplary embodiments, one or more electrodes are positioned on a head of the patient, at block 530. In some embodiments, the one or more electrodes comprise a plurality of electrodes positioned at different locations on a head of the subject, for example at locations C3, C4, Cz, FCz, P3, Pz and P4 of the 10-20 coordinate system, on a head of the subject, or at any combination of these locations. In some embodiments, the electrodes are attached to a head of the patient, for example to the scalp, using gel.

According to some exemplary embodiments, recording from the electrodes is initiated at block 532. In some embodiments, the electrodes are used to record EEG signals. In some embodiments, recording is initiated at block 532 before or after electrodes positioning at block 530.

According to some exemplary embodiments, contact of the electrodes with the head of the subject is determined at block 534. In some embodiments, the contact is determined based on the recording initiated at block 532. In some embodiments, contact of the electrodes with the head of the patient is presented to the supervisor or patient, for example using an interface showing electrodes with proper contact and electrodes that are not properly contacting the head of the patient. In some embodiments, the system alerts the supervisor and/or the user of which electrodes are not properly contacted during treatment and optionally what action to take to remedy the problem. In some embodiments, recording is initiated at block 532 after determining electrodes contact at block 534.

According to some exemplary embodiments, a treatment session optionally includes an eyes closed session, at block 536. In some embodiments, during the eyes close session signals are recorded from the patient when the patient is in a rest mode, for example to calibrate the recording and/or an EEG measurement system.

According to some exemplary embodiments, a treatment session optionally includes a global baseline session, at block 538. In some embodiments, during the global baseline session, EEG signals are measured and are collected for a pre-determined time period necessary to determine an activity of the at least one limbic brain network or a biomarker of the activity, for example using an EFP of the limbic brain network. In some embodiments, the pre-determined time period is up to 3 minutes, for example up to 2.5 minutes, up to 2 minutes, up to 1.5 minutes, up to 1 minute.

Optionally, a local baseline is calculated before every NF cycle. In some embodiments, during the local baseline session, EEG signals are measured and are collected for a pre-determined time period necessary to determine an activity of the at least one limbic brain network, for example using an EFP of the limbic brain network. In some embodiments, the pre-determined time period shorter than 2 minutes, for example shorter than 1.5 minute, shorter than 1 minute, or any intermediate, smaller or larger value.

According to some exemplary embodiments, a treatment session comprises one or more training cycles, at block 540. In some embodiments, the one or more training cycles comprise 1, 2, 3, 4, 5, 6, 7 or any larger number of NF training cycles. In some embodiments, the training cycles, for example the NF training cycles are consecutive training cycles. Optionally, the NF training cycles are continuously performed at block 540. Optionally, a delay between two consecutive NF training cycles is shorter than 2 minutes, for example shorter than 60 seconds, shorter than 30 seconds, shorter than 10 seconds, shorter than 5 seconds, shorter than 1 second or any intermediate, shorter or longer time period. Optionally, the training cycles comprise at least one transfer cycle 542, in which feedback regarding the activation level of the at least one limbic brain network was not delivered to the patient.

According to some exemplary embodiments, during the NF cycles, the patient watches a patient interface, for example a visual, an audio, a tactile and/or an olfactory interface which presents a scenario that changes according to the determined activity level of the at least one limbic brain network, while performing a task, for example a cognitive task and/or an emotional task. In some embodiments, the cognitive task is selected to increase the activity level of the at least one limbic brain network or a biomarker thereof. In some embodiments, an increase of the activity of the at least one limbic brain network or the biomarker thereof, increases or decreases the amount of stimulating cues delivered to the patient as part of the interface. Additionally, human detectable indications, for example visual, audio, tactile, and/or olfactory indications delivered to the patient while watching the interface indicate if a target activity level of the limbic brain network or biomarker thereof was crossed or if the determined activity is closer or is in a desired direction towards the target activity level.

### Exemplary EEG Processing

The model used at block 510 in FIG. 5A, was generated based on a large number of samples from many subjects, for example patients suffering from one or more PTSD symptoms, that participated in a NF treatment.

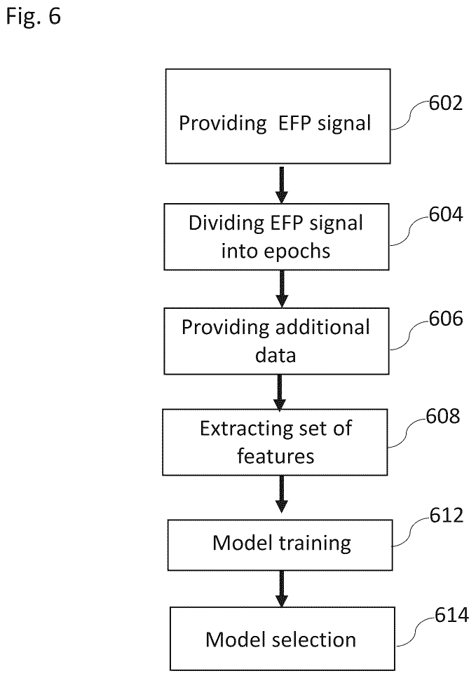

Reference is now made to FIG. 6 depicting a process for generating the model, according to some exemplary embodiments of the invention.

In some embodiments, electrical fingerprint (EFP) neuromodulation signals that are being generated during the NF treatment are provided at block 602. Optionally, an EEG signals are provided at block 602 instead of the EFP.

In some embodiments, the EFP signals or the EEG signals are divided into a plurality of epochs, at block 604. In some embodiments, when generating the model, the EFP or the EEG signal is divided into a plurality of epochs, each having a time period between 1 second-180 seconds, for example a time period between 30 seconds and 90 seconds, a time period between 50 seconds and 80 seconds, or any intermediate, smaller or larger time period.

Additional data is optionally provided at block 606, for example demographic data, EEG data, clinical data, physiological measurements, medical history, clinical data, accelerometer measurements, eye-tracking, cardiovascular measurements, etc.

A set of features was extracted from the provided EFP or EEG signals and optionally from the provided additional data, at block 608. In some embodiments, the set of features is extracted separately for each epoch or for a predetermined number of epochs of the provided EFP signal. The set of features includes one or more of, First Peak to peak score (extraction of this feature provides the peak-to-peak difference between the first positive peak point and the first negative peak point of the EFP signal. If no positive peak point can be identified prior to the negative peak point, then the difference between the first value of the EFP signal and the first negative peak point is returned), Max negative slope (extraction of this feature provides the largest negative linear slope coefficient that can be calculated within the limits of the EFP segment), NF slope (extraction of this feature provides the slope coefficient of a linear regression line that is fitted to the neurofeedback EFP segment), KL divergence (extraction of this feature provides the Kullback-Leibler divergence that represent the difference between local baseline and neurofeedback probability distributions), JS divergence (extraction of this feature provides the Jensen-Shannon divergence as an extended and symmetrical version of the KL divergence), Variance difference (extraction of this feature provides the Leven's test value for variance difference between the local baseline and neurofeedback segment), t-value (extraction of this feature provides Welch's t-test statistic value for the difference between the means of local baseline and neurofeedback segment), NF Kurtosis (extraction of this feature provides a Fisher's kurtosis of neurofeedback EFP distribution), NF Skewness (extraction of this feature provides a sample skewness of neurofeedback EFP distribution), AU LBL median (extraction of this feature provides a neurofeedback segment EFP area under the local baseline median), AU LBL mean (extraction of this feature provides a neurofeedback segment EFP area under the local baseline mean), Modified Z score (extraction of this feature provides a difference between the medians of local baseline and neurofeedback segment in terms of median absolute deviation of the local baseline), Modified Z score with mean (extraction of this feature provides a difference between the median of local baseline and the mean of the neurofeedback segment in terms of median absolute deviation of the local baseline), Z score with median (extraction of this feature provides a difference between the medians of local baseline and neurofeedback segment in terms of inter-quantile-range of the local baseline), Z score with mean median (extraction of this feature provides a difference between the median of the local baseline and the mean of the neurofeedback segment in terms of inter-quantile-range of the local baseline), Z score (extraction of this feature provides a difference between the means of local baseline and neurofeedback segment in terms of standard deviations of the local baseline), CL Effect size (extraction of this feature provides a probability of superiority of local baseline compared to neurofeedback segment EFP), Effect size (extraction of this feature returns a Cohen's d effect size representing the difference between local baseline and neurofeedback segment mean EFPs, divided by the pooled standard deviation), NF entropy (extraction of this feature provides a Shannon Entropy of the neurofeedback segment EFP, LBL entropy (extraction of this feature provides a Shannon Entropy of the local baseline EFP), NF std (extraction of this feature provides a neurofeedback segment standard deviation EFP), NF median (extraction of this feature provides a neurofeedback segment median EFP), NF mean (extraction of this feature provides a neurofeedback segment mean EFP), LBL std (extraction of this feature provides a local baseline standard deviation EFP), LBL median (extraction of this feature provides a local baseline median EFP), LBL mean (extraction of this feature provides a local baseline mean EFP), Part (the number of NF segment that is equivalent to LBL duration when NF and LBL are not equal in their duration. If NF and LBL are of the same duration, then there will be only 1 Part), Cycle (NF cycle number), and Session (NF session number), Pearson correlation coefficient (extraction of this feature provides a standardized measure for the linear correlation between the local baseline and neurofeedback segment EFP), Covariance coefficient (extraction of this feature provides an unstandardized measure of the joint variability of the local baseline and neurofeedback segment EFP), Granger causality (extraction of this feature provides the p-value of a statistical hypothesis test that determines whether the local baseline EFP is useful in forecasting the neurofeedback segment EFP), Reversed granger causality (extraction of this feature provides the p-value of a statistical hypothesis test that determines whether the neurofeedback segment EFP is useful in forecasting the local baseline EFP), Dynamic Time Warping (extraction of this feature provides a temporal-independent metric of similarity between the local baseline and neurofeedback segment EFP, Synchronization likelihood (extraction of this feature provides a metric of generalized synchronization that detects linear and non-linear dependencies between the local baseline and neurofeedback segment EFP, NF spectral centroid (extraction of this feature provides an indication of the spectral center of mass in the neurofeedback EFP signal), LBL spectral centroid (extraction of this feature provides an indication of the spectral center of mass in the local baseline EFP signal), NF spectral flatness (extraction of this feature provides a measure of Wiener entropy that represents the waveform noise of the neurofeedback EFP signal), LBL spectral flatness (extraction of this feature provides a measure of Wiener entropy that represents the waveform noise of the local baseline EFP signal).

The features were extracted to a tabular data format.

The model was trained at block 612, for example using Sequential attention based deep neural network with Transformer blocks that are tailored for tabular data, trained with self-supervised approach that utilize previously acquired unlabeled data (increases the overall dataset size from ~12 k to ~20 k). The model is trained to predict treatment success for multiple thresholds (e.g., 6-point/12-point improvement in clinical assessment), and multiple periods (e.g., 8 weeks after treatment onset/3 months post treatment). Multiple models are trained to broadly cover the cross-validation combinations.

The model was selected at block 614, for example based on treatment success prediction performance, optionally by examining the model on different clinical improvement thresholds and/or different post-treatment periods.

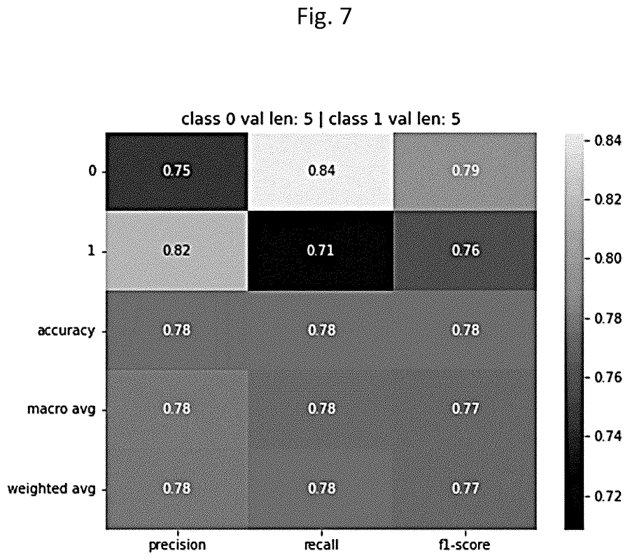

FIG. 7 shows an example of a heat map describing model accuracy scores.

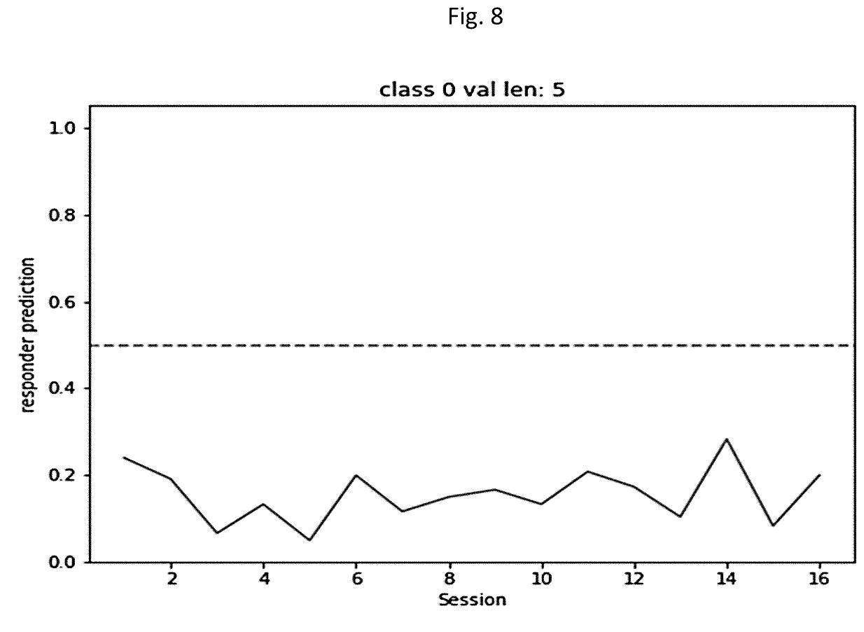

FIG. 8 shows a prediction score (from 0: unsuccessful to 1: successful treatment) for 5 patients that did not show clinical improvement post-treatment.

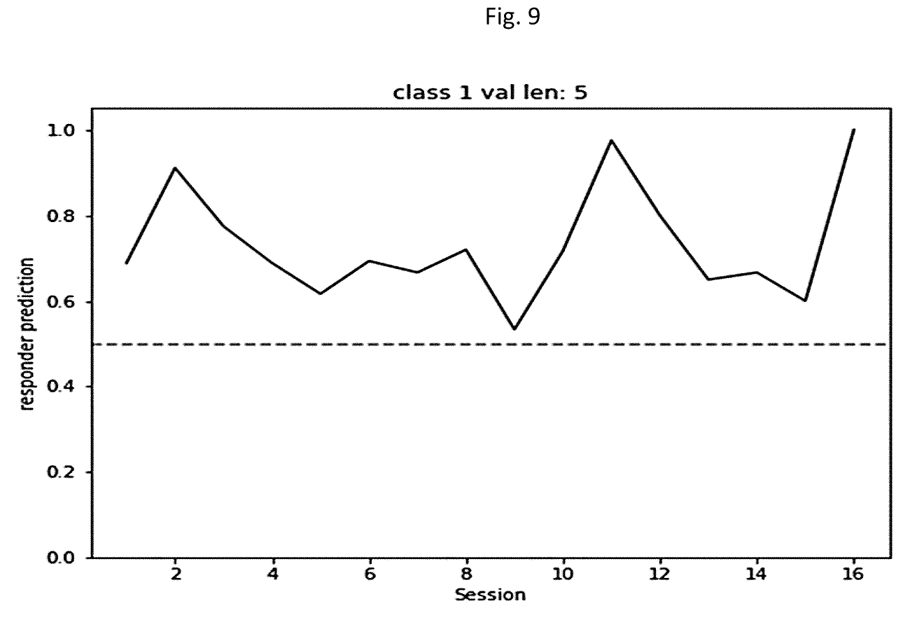

FIG. 9 shows a prediction score (from 0: unsuccessful to 1: successful treatment) for 5 patients that showed improvement post-treatment.

### Exemplary Usage of the Model:

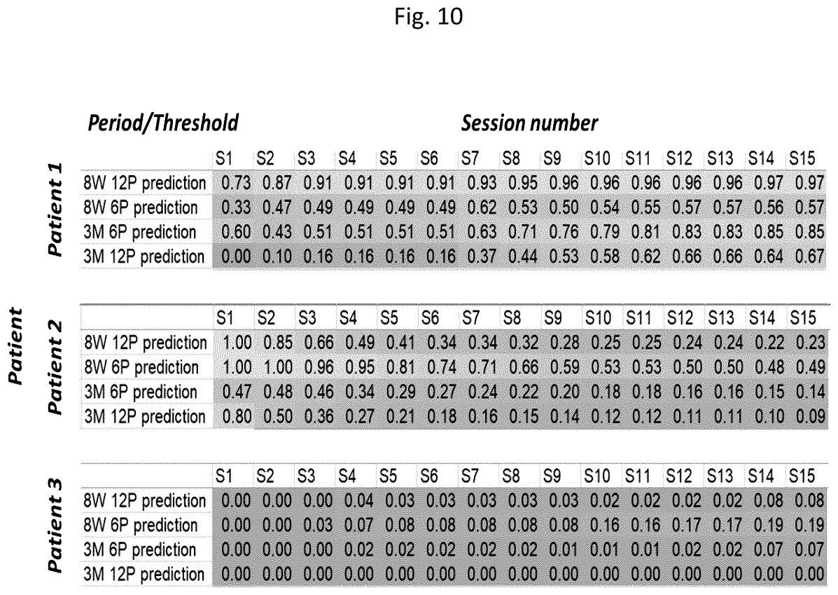

For each prediction point (i.e., at the end of each session or during the session), all available data until the prediction point is fed to the model to provide a more robust inference. For example, at the end of session number 2, the data of session 1 and 2 will be used for success prediction or alternatively only data from session 2 will be used. Accordingly, at the end of session 3 the data from session 1, 2 and 3 will be used for inference, etc. following this logic, the multi-threshold/multi-period scoring technique can be applied during training and the scores across prediction can guide protocol decision making (see for example FIG. 10).

FIG. 10 shows examples of multi-threshold and/or multi periods success predictions on three patients. The multi-threshold success predictions relate to prediction of success in lowering a score of CAPS-5 assessment questionnaire in 12 points (12P) or in 6 points (6P), relative to a baseline score. The multi time period success predictions relates to predicting of success of a patient to lower the CAPS-5 after 8 weeks of NF treatment (indicating immediate success of the NF treatment) or after 3 months from completing the treatment (indicating prolong effect of the NF treatment).

As shown in FIG. 10, patient 1 (top panel) is an example of a patient that had significant clinical improvement and received high prediction scores during treatment with increasing pattern as the treatment continues.

A patient may have a clinical improvement immediately following the treatment, for example after 8 weeks of treatment, but according to the prediction model his scores are expected to decrease after completion of the treatment, for example 3 months following the treatment. In this case, in some embodiments the system using the prediction model will provide suggestions to prolong an existing treatment protocol, for example by adding additional treatment sessions, or by modifying the protocol for example to prolong each or some treatment sessions.

Patient 2 (middle panel) is an example of a patient that had a clinical improvement only immediately after treatment (i.e., 8 W) but his scores had decreased on the 3M clinical assessment. Accordingly, the model indicates low scores for the 3M period and suggests that this patient might had been benefited from a prolonged treatment or a modified protocol or from additional treatment session, for example treatment boosters.

Patient 3 (Lower panel) is an example of a patient that did not show clinical improvement post treatment and indeed his prediction scores are low throughout treatment. The model results would suggest that a termination of treatment can be considered for this patient.

As used herein with reference to quantity or value, the term “about” means “within ±10% of”.

The terms “comprises”, “comprising”, “includes”, “including”, “has”, “having” and their conjugates mean “including but not limited to”.

The term “consisting of” means “including and limited to”.

The term “consisting essentially of” means that the composition, method or structure may include additional ingredients, steps and/or parts, but only if the additional ingredients, steps and/or parts do not materially alter the basic and novel characteristics of the claimed composition, method or structure.

As used herein, the singular forms “a”, “an” and “the” include plural references unless the context clearly dictates otherwise. For example, the term “a compound” or “at least one compound” may include a plurality of compounds, including mixtures thereof.

Throughout this application, embodiments of this invention may be presented with reference to a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as “from 1 to 6” should be considered to have specifically disclosed subranges such as “from 1 to 3”, “from 1 to 4”, “from 1 to 5”, “from 2 to 4”, “from 2 to 6”, “from 3 to 6”, etc.; as well as individual numbers within that range, for example, 1, 2, 3, 4, 5, and 6. This applies regardless of the breadth of the range.

Whenever a numerical range is indicated herein (for example “10-15”, “10 to 15”, or any pair of numbers linked by these another such range indication), it is meant to include any number (fractional or integral) within the indicated range limits, including the range limits, unless the context clearly dictates otherwise. The phrases “range/ranging/ranges between” a first indicate number and a second indicate number and “range/ranging/ranges from” a first indicate number “to”, “up to”, “until” or “through” (or another such range-indicating term) a second indicate number are used herein interchangeably and are meant to include the first and second indicated numbers and all the fractional and integral numbers therebetween.

Unless otherwise indicated, numbers used herein and any number ranges based thereon are approximations within the accuracy of reasonable measurement and rounding errors as understood by persons skilled in the art

As used herein the term “method” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts.

As used herein, the term “treating” includes abrogating, substantially inhibiting, slowing or reversing the progression of a condition, substantially ameliorating one or more clinical or aesthetical symptoms of a condition, for example a mental disorder or substantially preventing the appearance of clinical or aesthetical symptoms of a condition.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination or as suitable in any other described embodiment of the invention. Certain features described in the context of various embodiments are not to be considered essential features of those embodiments, unless the embodiment is inoperative without those elements.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

It is the intent of the applicant(s) that all publications, patents and patent applications referred to in this specification are to be incorporated in their entirety by reference into the specification, as if each individual publication, patent or patent application was specifically and individually noted when referenced that it is to be incorporated herein by reference. In addition, citation or identification of any reference in this application shall not be construed as an admission that such reference is available as prior art to the present invention. To the extent that section headings are used, they should not be construed as necessarily limiting. In addition, any priority document(s) of this application is/are hereby incorporated herein by reference in its/their entirety.

1-53. (canceled)

54. A system for calculating a prognosis predicting score, comprising:
a memory, wherein said memory stores a signal indicating activity of at least one specific brain network of a subject suffering from at least one symptom of a mental disorder or changes thereof, measured before, during and/or after a neurofeedback (NF) treatment delivered to said subject, and at least one prognosis prediction software;
a control circuitry, wherein said control circuitry is configured to:
process said stored activity indicating signal;
calculate a prognosis predicting score based on said processing results and using said at least one prognosis prediction software, wherein said prognosis predicting score indicates a predicted prognosis of a state of said subject at an end of said NF treatment and/or during said NF treatment.

55. A system according to claim 54, wherein said control circuitry is configured to process said stored activity indicating signal by extracting values of predetermined parameters from said stored activity indicating signal, and wherein said calculate comprises calculate said prognosis predicting score by using said extracted values as input information for said at least one prognosis prediction software.

56. A system according to claim 55, wherein said control circuitry is configured to process said stored activity indicating signal by dividing said activity signal into epochs within a range between 1 millisecond and 120 seconds and wherein said extracting comprises extracting said values of said activity indicating signal parameters independently for each epoch of said epochs.

57. A system according to claim 54, wherein said prognosis prediction score predicts an ability of said subject to achieve a target score in a clinical assessment exam that is used to measure a clinical state of patients suffering from said mental disorder, during, at an end and/or following said NF treatment, wherein said target score or indication thereof is stored in said memory.

58. A system according to claim 54, wherein said prognosis prediction score predicts an ability of said subject to achieve a target change in a score of a clinical assessment exam that is used to measure a clinical state of patients suffering from said mental disorder, relative to a baseline value, during, at an end and/or following said NF treatment, wherein at least one of said target change and/or said baseline value is stored in said memory.

59. A system according to claim 57, wherein said mental disorder comprises a stress disorder, and wherein said at least one specific brain network comprises a limbic system brain network, and wherein said clinical assessment exam comprises a Clinician-Administered PTSD Scale for DSM-5 (CAPS-5) and/or parameters or domains of the CAPS-5, a PTSD Checklist for DSM-5 (PCL-5), and a Patient Health Questionnaire-9 (PHQ-9).

60. A system according to claim 57, wherein said mental disorder comprises depression or major depressive disorder (MDD), and wherein said at least one specific brain network comprises a reward system brain network, and wherein said clinical assessment exam comprises at least one of, Hamilton Depression Rating Scale or any derivative, the Snaith-Hamilton Pleasure Scale (SHAPS) or any derivative, the Montgomery-Asberg Depression Rating Scale (MADRS) or derivatives, the Patient Health Questionnaire (PHQ) or derivatives, the Clinical Global Impression (CGI) or derivatives.

61. A system according to claim 54, wherein said control circuitry is configured to generate a prognosis prediction indication of said calculated prognosis predicting score.

62. A system according to claim 61, wherein said prognosis prediction indication comprises at least one suggestion to modify at least one parameter of said NF treatment based on said calculated prognosis predicting score, wherein said at least one suggestion is stored in said memory.

63. A system according to claim 62, wherein said at least one parameter comprises at least one of, number of treatment sessions, duration of at least one treatment session, and/or time interval between two treatment sessions.

64. A system according to claim 61, comprising:
a communication circuitry configured to communicate with at least one remote device;
wherein said control circuitry is configured to signal said communication circuitry to deliver said prognosis prediction indication to said remote device.

65. A method for predicting prognosis of a patient suffering from a mental disorder, undergoing a neurofeedback (NF) treatment, comprising:
measuring EEG signals from a brain of said patient, before, during and/or after at least one treatment session of said NF treatment;
generating an indicator of activity of at least one specific brain network based on said measured EEG signals;
processing said indicator activity;
predicting in a timed relation with said at least one treatment session, a prognosis of a state of said patient at an end of said NF treatment and/or during said NF treatment, based on said processed activity indicator.

66. A method according to claim 65, wherein said predicting prognosis comprises predicting an improvement in said patient state at an end of at least one session of said NF treatment.

67. A method according to claim 65, wherein said predicting prognosis comprises predicting an improvement in said patient state at least one week from completing said NF treatment.

68. A method according to claim 65, wherein said predicting prognosis comprises predicting improvement in one or more clinical assessment exams that is used to measure a clinical state of patients suffering from said mental disorder.

69. A method according to claim 68, wherein said predicting improvement comprises predicting an ability of said patient to achieve at least one of, a target score of said one or more clinical assessment exams, a target range of scores of said one or more clinical assessment exams.

70. A method according to claim 68, wherein said predicting improvement comprises predicting an ability of said patient to reach a target reduction in a score of said one or more clinical assessment exams compared to a reference score or a base line score, when performing said one or more clinical assessment exams.

71. A method according to claim 68, wherein said mental disorder comprises post-traumatic stress disorder (PTSD), and wherein said predicting improvement comprises predicting an ability of said subject to reach a reduction of at least 3 points in a Clinician-Administered post-traumatic stress disorder (PTSD) Scale for DSM-5 (CAPS-5), compared to a reference or a baseline score of said CAPS-5, and/or a reduction of at least 3 points in a PTSD Checklist for DSM-5 (PCL-5), compared to a reference or a baseline score of said PCL-5.

72. A method according to claim 68, wherein said mental disorder comprises depression or major depressive disorder (MDD), and wherein said predicting improvement comprises predicting an ability of said subject to reach a reduction of at least 3 points in a Hamilton Depression Rating Scale (HDRS), compared to a reference or a bassline score of said HDRS, and/or a reduction of at least 4 points in a Snaith-Hamilton Pleasure Scale (SHAPS) compared to a reference or a bassline score of said SHAPS, and/or a reduction of at least 6 points in a Montgomery-Åsberg Depression Rating Scale (MADRS) compared to a reference or a bassline score of said MADRS.

73. A method according to claim 68, wherein said mental disorder comprises Anhedonia, and wherein said predicting improvement comprises predicting an ability of said subject to reach a reduction of at least 5 points in a Snaith-Hamilton Pleasure Scale (SHAPS) compared to a reference or a bassline score of said SHAPS.
